(19) United States
(12) Patent Application Publication
(10) Pub. No.: US 2025/0372256 A1
(43) Pub. Date: Dec. 4, 2025

(54) ANCESTRY-RELATED KRAS CO-ALTERATION PATTERNS AS PROGNOSTIC BIOMARKERS

(71) Applicants: Foundation Medicine, Inc., Boston, MA (US); Genentech, Inc., South San Francisco, CA (US)

(72) Inventors: Saumya Dushyant SISOUDIYA, Rockville, MD (US); Ethan S. SOKOL, Somerville, MA (US); Smruthy K. SIVAKUMAR, Everett, MA (US); Zhen SHI, South San Francisco, CA (US); Gaurav PATHRIA, South San Francisco, CA (US)

(73) Assignees: Foundation Medicine, Inc., Boston, MA (US); Genentech, Inc., South San Francisco, CA (US)

(21) Appl. No.: 19/224,407

(22) Filed: May 30, 2025

Related U.S. Application Data
(60) Provisional application No. 63/655,914, filed on Jun. 4, 2024.

(51) Int. Cl. G16H 50/20; G16B 20/20

(52) U.S. Cl. CPC G16H 50/20; G16B 20/20

(57) ABSTRACT

Biomarker-based methods for predicting disease prognosis and treatment outcomes are described. In some instances, the disclosed methods can comprise, for example, detecting in a sample from the subject, a KRAS gene alteration; detecting, in the sample from the subject, at least one of a STK11 gene alteration or a KEAP1 gene alteration; and predicting a prognosis and/or treatment outcome for the subject based on the detection of a co-alteration of the KRAS gene and at least one of the STK11 and/or KEAP1 genes. In some instances, the disease may be cancer, e.g., non-squamous non-small cell lung cancer (NSCLC). In some instances, the prediction of treatment outcomes may comprise prediction of treatment outcomes when treating a cancer (e.g., NSCLC) with an immune checkpoint inhibitor (ICI) or a KRAS G12C inhibitor.

### CROSS-REFERENCE TO RELATED APPLICATIONS

This application claims the priority benefit of U.S. Provisional Patent Application Ser. No. 63/655,914, filed Jun. 4, 2024, the contents of which are incorporated herein by reference in their entirety.

### FIELD

The present disclosure relates generally to methods for predicting cancer prognosis and treatment outcomes, and more specifically to biomarkers for predicting prognosis and treatment outcomes for cancers such as non-squamous non-small cell lung cancer (non-SQ NSCLC).

### BACKGROUND

Lung cancer is the second most commonly diagnosed cancer globally and is the leading cause of cancer-related deaths. Non-Small Cell Lung Cancer (NSCLC) accounts for 80-85% of all lung cancers, with non-squamous (non-Sq) NSCLC, particularly lung adenocarcinoma, as the most common histologic subtype. Despite declines in NSCLC-related mortality in the past decade in the US, racial and ethnic disparities persist, including minority patient groups presenting with cancer at a younger age and more advanced stage compared to White individuals.

The advent of precision medicine and comprehensive genomic profiling using targeted sequencing has led to unique opportunities to identify the optimal treatment options for patients, requiring a growing emphasis on biomarker-driven clinical trials and a need to better define patient cohorts for these trials. As such, current drug development and patient enrollment in clinical trials are heavily influenced by our understanding of the prevalence of genomic alterations. However, due to the limited access to certain medical centers by minority groups and other historical biases, the databases utilized to inform the size of biomarker-selected patient populations are overwhelmingly composed of data from patients of Western European descent. Further, minority populations including Black, Asian, Hispanic/Latino, and Indigenous Peoples represent a small fraction of the patient population characterized in the widely utilized dataset, The Cancer Genome Atlas (TCGA) (12%, 3%, 3%, <0.5% respectively), and these populations continue to be underrepresented in clinical trials.

Genomic profiles, gene expression changes and prognostic significance of specific biomarkers have been shown to vary by race and ancestry across different tumor types, including NSCLC. However, interpretation of clinical trial outcomes and real-world implications, including identification of predictive and prognostic biomarkers, is heavily constrained in underrepresented populations. Due to a limited understanding of biomarker prevalence in patients from minority populations, forecasting clinical trial enrollment metrics to facilitate inclusive and equitable healthcare has also been hampered. Furthermore, because of their underrepresentation in clinical studies, fuller comprehension of the implications of the clinical data, including therapeutic indexes, pharmacokinetics and pharmacodynamics and drug safety and toxicity attributes of experimental treatments in the minority patient population groups has been challenging.

There are many contributing factors to the disparities observed in cancer outcomes and representation in clinical studies, including a general lack of trust in the medical establishment, limited awareness of cancer screening and clinical trial opportunities, long-standing effects of structural racism and environmental factors across different racial/ethnic groups. However, the magnitude of observed differences cannot solely be attributed to socioeconomic factors. Differences in the prevalence and the landscape of molecular alterations in different ancestry groups may also impact cancer outcomes and impact equitable representation in clinical trials. However, the continued lack of diversity in clinical studies has led to a rather poor understanding of the contribution of genomics to the disparities in the prevalence and outcomes of non-Sq NSCLC in different ancestry groups. Thus improved methods for predicting cancer prognosis and treatment outcomes (e.g., for non-Sq NSCLC) that account for potential differences in the landscape of molecular alterations in different ancestry groups are required.

### BRIEF SUMMARY OF THE INVENTION

Improved methods of diagnosing disease, predicting disease prognosis, selecting a disease treatment, adjusting a disease treatment dose, monitoring disease progression or recurrence, and/or identifying a subject for inclusion in a clinical trial, based on a biomarker comprising the detection of a co-alteration of the KRAS gene and at least one of the STK11 and/or KEAP1 genes, are described. The disclosed methods account for differences in the landscape of molecular alterations in different ancestry groups, and thus provide more accurate predictions of disease prognosis and disease treatment outcomes (e.g., cancer prognosis and cancer treatment outcomes).

Disclosed herein are methods for diagnosing or confirming a diagnosis of disease in a subject, the methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; detecting, using the one or more processors, a KRAS gene alteration in the sample from the subject based on the sequence read data; detecting, using the one or more processors, at least one of a STK11 gene alteration or a KEAP1 gene alteration in the sample from the subject based on the sequence read data; and diagnosing or confirming a diagnosis of disease in the subject based on the detection of a co-alteration of the KRAS gene and at least one of the STK11 and/or KEAP1 genes.

In some embodiments, the method further comprises: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.

Disclosed herein are methods for predicting a treatment outcome for a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and predicting a treatment outcome for the subject based on the detection of a co-alteration of the KRAS gene and at least one of the STK11 and/or KEAP1 genes.

In some embodiments, the disease is cancer, and optionally, wherein the cancer is non-squamous non-small cell lung cancer (NSCLC).

In some embodiments, the predicted outcome for the subject is poor compared to that for a sample in which only a KRAS gene alteration is detected.

In some embodiments, the method further comprises detecting at least one biomarker of genetic ancestry in the sample from the subject, and adjusting the predicted treatment outcome based on the detection of the at least one biomarker of genetic ancestry. In some embodiments, the at least one biomarker of genetic ancestry is indicative of European, African, East Asian, South Asian, and admixed American ancestry. In some embodiments, the at least one biomarker of genetic ancestry comprises a single nucleotide polymorphism (SNP)-based biomarker.

In some embodiments, the method further comprises detecting a co-occurrence of a KRAS gene alteration and a GNAS gene alteration in a sample from a subject for which the at least one biomarker of genetic ancestry is indicative of admixed American ancestry, and adjusting the predicted treatment outcome based on the detection of the GNAS gene alteration.

In some embodiments, the method further comprises detecting a co-occurrence of a KRAS gene alteration and an ARID1A gene alteration in a sample from a subject for which the at least one biomarker of genetic ancestry is indicative of South Asian ancestry, and adjusting the predicted treatment outcome based on the detection of the ARID1A gene alteration.

In some embodiments, the method further comprises a tumor mutational burden (TMB) in a sample from a subject for which the at least one biomarker of genetic ancestry is indicative of African, and adjusting the predicted treatment outcome based on the determined TMB.

In some embodiments, the KRAS gene alteration comprises a KRAS short variant, a KRAS gene amplification, or any combination thereof. In some embodiments, the KRAS gene alteration comprises a KRAS G12C alteration.

In some embodiments, the STK11 and/or KEAP1 gene alterations comprise loss-of-function alterations.

In some embodiments, the disease is cancer. In some embodiments, the cancer is non-squamous non-small cell lung cancer (NSCLC).

In some embodiments, treatment of the disease comprises treatment with an immune checkpoint inhibitor (ICI). In some embodiments, treatment of the disease comprises treatment with a KRAS G12C inhibitor.

Also disclosed herein are systems comprising: one or more processors; and a memory communicatively coupled to the one or more processors and configured to store instructions that, when executed by the one or more processors, cause the system to: receive sequence read data for a plurality of sequence reads derived from a sample from a subject; detect a KRAS gene alteration in the sample from the subject based on the sequence read data; detect at least one of a STK11 gene alteration or a KEAP1 gene alteration in the sample from the subject based on the sequence read data; and diagnose or confirm a diagnosis of disease in the subject based on the detection of a co-alteration of the KRAS gene and at least one of the STK11 and/or KEAP1 genes.

Also disclosed herein are non-transitory computer-readable storage media storing one or more programs, the one or more programs comprising instructions, which when executed by one or more processors of a system, cause the system to: receive sequence read data for a plurality of sequence reads derived from a sample from a subject; detect a KRAS gene alteration in the sample from the subject based on the sequence read data; detect at least one of a STK11 gene alteration or a KEAP1 gene alteration in the sample from the subject based on the sequence read data; and diagnose or confirm a diagnosis of disease in the subject based on the detection of a co-alteration of the KRAS gene and at least one of the STK11 and/or KEAP1 genes.

Disclosed herein are methods for diagnosing or confirming a diagnosis of disease in a subject, the methods comprising: detecting, in a sample from the subject, a KRAS gene alteration; detecting, in the sample from the subject, at least one of a STK11 gene alteration or a KEAP1 gene alteration; and diagnosing or confirming a diagnosis of disease in the subject based on the detection of a co-alteration of the KRAS gene and at least one of the STK11 and/or KEAP1 genes.

Disclosed herein are methods for identifying a subject for treatment of a disease, the methods comprising: detecting, in a sample from the subject, a KRAS gene alteration; detecting, in the sample from the subject, at least one of a STK11 gene alteration or a KEAP1 gene alteration; and identifying the subject for treatment of the disease based on the detection of a co-alteration of the KRAS gene and at least one of the STK11 and/or KEAP1 genes.

Disclosed herein are methods for predicting a prognosis for a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and predicting a prognosis for the subject based on the detection of a co-alteration of the KRAS gene and at least one of the STK11 and/or KEAP1 genes.

Disclosed herein are methods for predicting a treatment outcome for a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and predicting a treatment outcome for the subject based on the detection of a co-alteration of the KRAS gene and at least one of the STK11 and/or KEAP1 genes. In some embodiments, the predicted outcome for the subject is poor compared to that for a sample in which only a KRAS gene alteration is detected.

In some embodiments, the method further comprises detecting at least one biomarker of genetic ancestry in the sample from the subject, and adjusting the predicted treatment outcome based on the detection of the at least one biomarker of genetic ancestry. In some embodiments, the at least one biomarker of genetic ancestry is indicative of European, African, East Asian, South Asian, and admixed American ancestry. In some embodiments, the at least one biomarker of genetic ancestry comprises a single nucleotide polymorphism (SNP)-based biomarker. In some embodiments, the method further comprises detecting a co-occurrence of a KRAS gene alteration and a GNAS gene alteration in a sample from a subject for which the at least one biomarker of genetic ancestry is indicative of admixed American ancestry, and adjusting the predicted treatment outcome based on the detection of the GNAS gene alteration. In some embodiments, the method further comprises detecting a co-occurrence of a KRAS gene alteration and an ARID1A gene alteration in a sample from a subject for which the at least one biomarker of genetic ancestry is indicative of South Asian ancestry, and adjusting the predicted treatment outcome based on the detection of the ARID1A gene alteration. In some embodiments, the method further comprises a tumor mutational burden (TMB) in a sample from a subject for which the at least one biomarker of genetic ancestry is indicative of African, and adjusting the predicted treatment outcome based on the determined TMB.

Disclosed herein are methods for selecting a treatment for a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and responsive to the detection of a co-alteration of the KRAS gene and at least one of the STK11 and/or KEAP1 genes, selecting the treatment for the subject.

Disclosed herein are methods for treating a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and responsive to the detection of a co-alteration of the KRAS gene and at least one of the STK11 and/or KEAP1 genes, treating the subject.

Disclosed herein are methods for adjusting a treatment dose for a subject diagnosed with or suspected of having a disease, the methods comprising: detecting, in a sample from the subject, a KRAS gene alteration; detecting, in the sample from the subject, at least one of a STK11 gene alteration or a KEAP1 gene alteration; and responsive to the detection of a co-alteration of the KRAS gene and at least one of the STK11 and/or KEAP1 genes, adjusting a treatment dose for the subject.

Disclosed herein are methods for identifying a subject diagnosed with or suspected of having a disease for inclusion in a clinical trial, the methods comprising: detecting, in a sample from the subject, a KRAS gene alteration; detecting, in the sample from the subject, at least one of a STK11 gene alteration or a KEAP1 gene alteration; and identifying a subject for inclusion in a clinical trial based on the detection of a co-alteration of the KRAS gene and at least one of the STK11 and/or KEAP1 genes.

Disclosed herein are methods for monitoring disease progression or recurrence in a subject comprising: detecting, in a first sample from the subject collected at a first timepoint, a co-alteration of a KRAS gene and at least one of a STK11 and/or KEAP1 genes; and detecting, in a second sample from the subject collected at a second timepoint, a co-alteration of a KRAS gene and at least one of a STK11 and/or KEAP1 genes; thereby monitoring the disease progression or recurrence. In some embodiments, the first time point is before the subject has been administered a disease treatment, and wherein the second time point is after the subject has been administered the disease treatment.

In any of the embodiments described herein, the KRAS gene alteration may comprise a KRAS short variant, a KRAS gene amplification, or any combination thereof. In some embodiments, the KRAS gene alteration comprises a KRAS G12C alteration. In some embodiments, the STK11 and/or KEAP1 gene alterations comprise loss-of-function alterations.

In any of the embodiments described herein, the disease may be cancer. In some embodiments, the cancer is non-squamous non-small cell lung cancer (NSCLC).

In any of the embodiments described herein, treatment of the disease can comprise treatment with an immune checkpoint inhibitor (ICI). In any of the embodiments described herein, treatment of the disease can comprise treatment with a KRAS G12C inhibitor.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for diagnosing or confirming a diagnosis of disease, in accordance with one embodiment of the present disclosure.

FIG. 2 provides a non-limiting example of a process flowchart for diagnosing or confirming a diagnosis of disease, in accordance with one embodiment of the present disclosure.

FIG. 3 provides a non-limiting example of a process flowchart for predicting a treatment outcome for a subject, in accordance with one embodiment of the present disclosure.

FIG. 4 depicts an exemplary computing device or system in accordance with one embodiment of the present disclosure.

FIG. 5 depicts an exemplary computer system or computer network, in accordance with some instances of the systems described herein.

FIG. 6A provides a non-limiting example of data for the overall KRAS alteration prevalence (top) and the prevalence of individual KRAS alterations (bottom) in each of five different ancestry groups.

FIG. 6B provides a non-limiting example of pie charts displaying the breakdown of the different KRAS alterations observed in each of five different ancestry groups (EUR: European, AFR: African, EAS: East Asian, SAS: South Asian, AMR: Admixed American).

FIG. 6C provides a non-limiting example of the overall EGFR alteration prevalence (top) and the prevalence of individual EGFR alterations (bottom) in each of five different ancestry groups.

FIG. 6D provides a non-limiting example of pie charts displaying the breakdown of the different EGFR alterations observed in each of five different ancestry groups.

FIG. 7A provides a non-limiting example of a tileplot showing the overall mutational spectrum of KRAS-altered non-Sq NSCLC, with alterations in the top 30 most frequently occurring genes displayed.

FIG. 7B provides a non-limiting example of data illustrating the comparative prevalence of co-occurring gene alterations in KRAS-altered non-Sq NSCLC based on ancestry.

FIG. 7C provides a non-limiting example of data illustrating patterns of co-occurrence and mutual exclusivity between KRAS and other gene alterations in each of five different ancestry groups.

FIG. 8A provides a non-limiting example of a tileplot showing the overall mutational spectrum of EGFR-altered non-Sq NSCLC, with alterations in the top 30 most frequently occurring genes displayed.

FIG. 8B provides a non-limiting example of data illustrating the comparative prevalence of co-occurring gene alterations in EGFR-altered non-Sq NSCLC based on ancestry.

FIG. 8C provides a non-limiting example of data illustrating patterns of co-occurrence and mutual exclusivity between EGFR and other gene alterations in each of five different ancestry groups.

FIG. 9A provides a non-limiting example of data illustrating patterns of tumor mutational burden in the overall cohort, KRAS-altered and EGFR-altered non-Sq NSCLC based on ancestry.

FIG. 9B provides a non-limiting example of data illustrating patterns of PD-L1 positivity from DAKO 22C3 in the overall cohort, KRAS-altered and EGFR-altered non-Sq NSCLC based on ancestry.

FIG. 10 provides a non-limiting example of a barplot showing the prevalence of the top gene alterations across different ancestry subgroups in the overall non-Sq NSCLC cohort based on ancestry.

FIG. 11A provides a non-limiting example of data illustrating the breakdown of cases with KRAS short variants only, amplifications only, and cases with both classes of alterations within each ancestry group.

FIG. 11B provides a non-limiting example of data illustrating the breakdown of cases with EGFR short variants only, amplifications only, and cases with both classes of alterations within each ancestry group.

FIG. 12 provides a non-limiting example of a barplot displaying the prevalence of cases, as a percentage of total cases (N), with co-occurring alterations in KRAS, KEAP1, and STK11 in each ancestry group (EUR: European, AFR: African, EAS: East Asian, SAS: South Asian, AMR: Admixed American).

FIG. 13A provides a non-limiting example of data illustrating patterns of co-occurrence and mutual exclusivity between KRAS short variants and other gene alterations in each of five different ancestry groups.

FIG. 13B provides a non-limiting example of data illustrating patterns of co-occurrence and mutual exclusivity between KRAS amplifications and other gene alterations in each of five different ancestry groups.

FIG. 14A provides a non-limiting example of data illustrating patterns of co-occurrence and mutual exclusivity between EGFR short variants and other gene alterations in each of five different ancestry groups.

FIG. 14B provides a non-limiting example of data illustrating patterns of co-occurrence and mutual exclusivity between EGFR amplifications and other gene alterations in each of five different ancestry groups.

### DETAILED DESCRIPTION

Improved methods of diagnosing disease, predicting disease prognosis, selecting a disease treatment, adjusting a disease treatment dose, monitoring disease progression or recurrence, and/or identifying a subject for inclusion in a clinical trial, based on a biomarker comprising the detection of a co-alteration of the KRAS gene and at least one of the STK11 and/or KEAP1 genes, are described. The disclosed methods account for differences in the landscape of molecular alterations in different ancestry groups, and thus provide more accurate predictions of disease prognosis and disease treatment outcomes (e.g., cancer prognosis and cancer treatment outcomes).

In some instances, for example, the disclosed methods may comprise: detecting a KRAS gene alteration in the sample from a subject (e.g., based on sequence read data or other genotyping data); detecting at least one of a STK11 gene alteration or a KEAP1 gene alteration in the sample from the subject (e.g., based on the sequence read data or other genotyping data); and diagnosing or confirming a diagnosis of disease in the subject, identifying the subject for treatment of a disease, predicting a prognosis for the subject, selecting a treatment for the subject, treating the subject, adjusting a treatment dose for the subject, identifying the subject for inclusion in a clinical trial, and/or monitoring the disease progression or recurrence in the subject, based on the detection of a co-alteration of the KRAS gene and at least one of the STK11 and/or KEAP1 genes.

In some instances, the disease may be cancer. In some instances, the cancer may be non-squamous non-small cell lung cancer (NSCLC).

In some instances, treatment of the disease may comprise treatment with an immune checkpoint inhibitor (ICI). In some instances, treatment of the disease may comprise treatment with a KRAS G12C inhibitor.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” is a computationally generated sequence generated by a sequencer to represent a sequence of bases of a single strand of a sequenced fragment. A sequence read can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at the 5′ and/or 3′ end), or any combination thereof.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Ancestry-Related KRAS Co-Alteration Patterns as Prognostic Biomarkers

The prevalence and co-alteration landscape of genomic alterations in non-Sq NSCLC was investigated using a diverse real-world cohort comprising patients of European (EUR), African (AFR), East Asian (EAS), South Asian (SAS) and Admixed American (AMR) ancestries. The analysis was focused on tumors with alterations in KRAS and EGFR, the two major oncogenic drivers of non-Sq NSCLC. Additionally, ancestry-associated patterns of programmed death-ligand 1 (PD-L1) expression and tumor mutational burden (TMB), known predictive biomarkers of response to immune checkpoint inhibitors, were investigated across the five ancestry groups to gain molecular insights into ancestry-based genomic landscapes as well as to better inform strategies for patient treatment and clinical trial enrollment.

Several ancestry-specific co-alterations in patients with non-squamous NSCLC were identified, including co-occurrence of KRAS and GNAS alterations in AMR, co-occurrence of KRAS and ARID1A alterations in SAS, and the mutual exclusivity of KRAS and NF1 alterations in the EUR and AFR ancestries. Contrastingly, EGFR-altered tumors exhibited a more conserved co-alteration landscape across ancestries. Patients of AFR ancestry exhibited the highest tumor mutational burden compared to other ancestry groups, with potential therapeutic implications for these tumors.

STK11 and KEAP1 alterations were identified as potential biomarkers for prediction of poor prognosis in NSCLC. The prevalence of STK11, KEAP1 alterations in patients with KRAS-mutant non-squamous NSCLC across different ancestry groups was previously understudied. This work provides these alteration rates. The significantly lower co-alteration rate of STK11 and KEAP1 in KRAS-altered EAS and AMR as compared to other ancestries raises questions regarding potential clinical implications, including treatment outcomes with IC1 and the recently emerging KRAS G12C inhibitors.

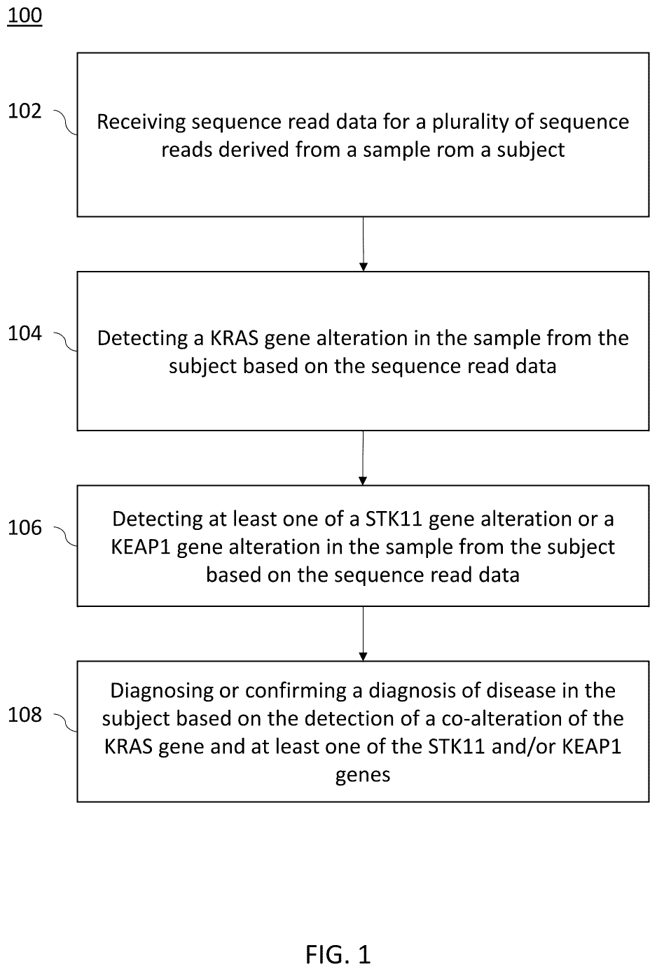

FIG. 1 provides a non-limiting example of a flowchart for a process 100 for diagnosing or confirming a diagnosis of disease. In some instances, all or a portion of process 100 may be performed as a computer-implemented method. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, sequence read data for a plurality of sequence reads derived from a sample from a subject are received (e.g., by one or more processors of a system configured to perform process 100).

In some instances, the sequence read is derived from a plurality of sequence reads that map to a plurality of genes or genomic loci. In some instances, for example, the plurality of genes or genomic loci may comprise at least 4, at least 6, at least 8, at least 10, at least 20, at least 30, at least 40, at least 50, at least 60, at least 70, at least 80, at least 90, at least 100, at least 200, at least 300, at least 400, at least 500, or more than 500 genes or genomic loci.

In some instances, the sequence read data may be derived by sequencing nucleic acids extracted from the sample using any of a variety of techniques known to those of skill including, but not limited to, next generation sequencing techniques (e.g., whole genome sequencing (WGS), whole exome sequencing (WES), and targeted sequencing).

In some instances, the sample may comprise a tissue biopsy sample, a liquid biopsy sample, or a normal control. In some instances, the sample may be a liquid biopsy sample and may comprise, e.g., blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof. In some instances, the sample may be a cancer specimen.

At step 104 in FIG. 1, a KRAS gene alteration—if present—is detected in the sample from the subject based on the sequence read data.

At step 106 in FIG. 1, at least one of a STK11 gene alteration or a KEAP1 gene alteration are detected—if present—in the sample from the subject based on the sequence read data.

In some instances, a gene alteration (e.g., a KRAS gene alteration, a STK11 gene alteration, or a KEAP1 gene alteration) may comprise a single nucleotide variant (SNV) (e.g., a single nucleotide substitution, a single nucleotide insertion, or a single nucleotide deletion), a short variant, an insertion or deletion (indel), a copy number variant (CNV), a rearrangement, a fusion, or a combination thereof.

In some instances, the KRAS gene alteration may comprise a KRAS short variant, a KRAS gene amplification, or a combination thereof. In some instances, the KRAS gene alteration may comprise a KRAS G12C alteration. In some instances, the STK11 and/or KEAP1 gene alterations may comprise a loss-of-function alteration.

Methods for identifying variant sequences based on, e.g., a comparison of sequence read data to a reference genome sequence, are known in the art (see, e.g., US Patent Application Publication No. 2023/0030656, which is incorporated herein by reference in its entirety), and are discussed in more detail elsewhere herein.

At step 108 in FIG. 1, a diagnosis (or confirmation of a diagnosis) of disease is made for the subject based on the detection of a co-alteration of the KRAS gene and at least one of the STK11 and/or KEAP1 genes.

In some instances, the process may further comprise detecting at least one biomarker of genetic ancestry in the sample from the subject, and adjusting the predicted treatment outcome based on the detection of the at least one biomarker of genetic ancestry. In some instances, the at least one biomarker of genetic ancestry is indicative of European, African, East Asian, South Asian, and admixed American ancestry. In some instances, the at least one biomarker of genetic ancestry comprises a single nucleotide polymorphism (SNP)-based biomarker.

In some instances, the process may further comprise detecting a co-occurrence of a KRAS gene alteration and a GNAS gene alteration in a sample from a subject for which the at least one biomarker of genetic ancestry is indicative of admixed American ancestry, and adjusting the diagnosis or confirmation of diagnosis based on the detection of the GNAS gene alteration.

In some instances, the process may further comprise detecting a co-occurrence of a KRAS gene alteration and an ARID1A gene alteration in a sample from a subject for which the at least one biomarker of genetic ancestry is indicative of South Asian ancestry, and adjusting the diagnosis or confirmation of diagnosis based on the detection of the ARID1A gene alteration.

In some instances, the process may further comprise a tumor mutational burden (TMB) in a sample from a subject for which the at least one biomarker of genetic ancestry is indicative of African, and adjusting the diagnosis or confirmation of diagnosis based on the determined TMB.

In some instances, the disease may be cancer. In some instances, the cancer may be non-squamous non-small cell lung cancer (non-Sq NSCLC).

In some instances, treatment of the disease may comprise treatment with an immune checkpoint inhibitor (ICI). In some instances, treatment of the disease may comprise treatment with a KRAS G12C inhibitor.

In some instances, in addition to, or instead of, diagnosing or confirming a diagnosis of disease, the method may comprise: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.

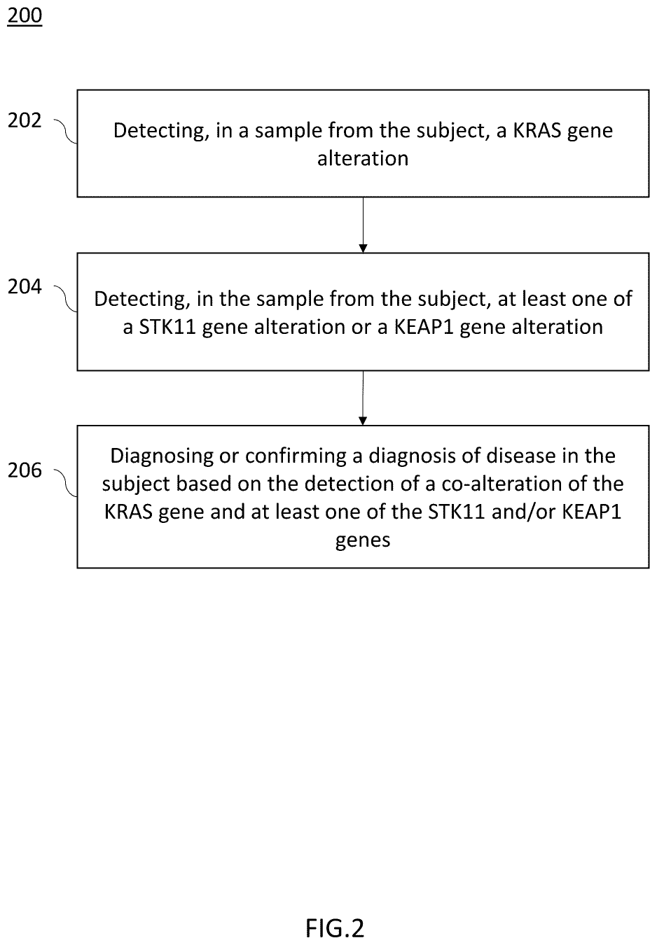

FIG. 2 provides a non-limiting example of a flowchart for a process 200 for diagnosing or confirming a diagnosis of disease. In some instances, the methods disclosed herein may be performed using sequencing-based and/or non-sequencing-based methods (e.g., microarray-based or PCR-based genotyping methods) for detection of KRAS, STK11, and KEAP1 gene alterations.

At step 202 in FIG. 2, a KRAS gene alteration-if present-is detected in the sample.

At step 204 in FIG. 2, at least one of a STK11 gene alteration or a KEAP1 gene alteration are detected—if present—in the sample.

In some instances, the detection of a KRAS gene alteration, a SYK11 gene alterations, and/or a KEAP1 gene alteration in the sample from the subject may comprise the use of a sequencing-based and/or non-sequencing-based method (e.g., a microarray-based or PCR-based genotyping method; see, e.g., Steuerwald et al. (2024), “Understanding the Biology and Testing Techniques for Pharmacogenomics in Oncology: A Practical Guide for the Clinician”, JCO Oncol. Pract. 20:1441-1461).

As noted above with respect to FIG. 1, in some instances, a gene alteration (e.g., a KRAS gene alteration, a STK11 gene alteration, or a KEAP1 gene alteration) may comprise a single nucleotide variant (SNV) (e.g., a single nucleotide substitution, a single nucleotide insertion, or a single nucleotide deletion), a short variant, an insertion or deletion (indel), a copy number variant (CNV), a rearrangement, a fusion, or a combination thereof.

In some instances, the KRAS gene alteration may comprise a KRAS short variant, a KRAS gene amplification, or a combination thereof. In some instances, the KRAS gene alteration may comprise a KRAS G12C alteration. In some instances, the STK11 and/or KEAP1 gene alterations may comprise a loss-of-function alteration.

At step 206 in FIG. 2, a diagnosis (or confirmation of a diagnosis) of disease is made for the subject based on the detection of a co-alteration of the KRAS gene and at least one of the STK11 and/or KEAP1 genes.

In some instances, the process may further comprise detecting at least one biomarker of genetic ancestry in the sample from the subject, and adjusting the predicted treatment outcome based on the detection of the at least one biomarker of genetic ancestry. In some instances, the at least one biomarker of genetic ancestry is indicative of European, African, East Asian, South Asian, and admixed American ancestry. In some instances, the at least one biomarker of genetic ancestry comprises a single nucleotide polymorphism (SNP)-based biomarker.

In some instances, the process may further comprise detecting a co-occurrence of a KRAS gene alteration and a GNAS gene alteration in a sample from a subject for which the at least one biomarker of genetic ancestry is indicative of admixed American ancestry, and adjusting the diagnosis or confirmation of diagnosis based on the detection of the GNAS gene alteration.

In some instances, the process may further comprise detecting a co-occurrence of a KRAS gene alteration and an ARID1A gene alteration in a sample from a subject for which the at least one biomarker of genetic ancestry is indicative of South Asian ancestry, and adjusting the diagnosis or confirmation of diagnosis based on the detection of the ARID1A gene alteration.

In some instances, the process may further comprise a tumor mutational burden (TMB) in a sample from a subject for which the at least one biomarker of genetic ancestry is indicative of African, and adjusting the diagnosis or confirmation of diagnosis based on the determined TMB.

In some instances, the disease may be cancer. In some instances, the cancer may be non-squamous non-small cell lung cancer (non-Sq NSCLC).

In some instances, treatment of the disease may comprise treatment with an immune checkpoint inhibitor (ICI). In some instances, treatment of the disease may comprise treatment with a KRAS G12C inhibitor.

In some instances, in addition to, or instead of, diagnosing or confirming a diagnosis of disease, the method may comprise: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.

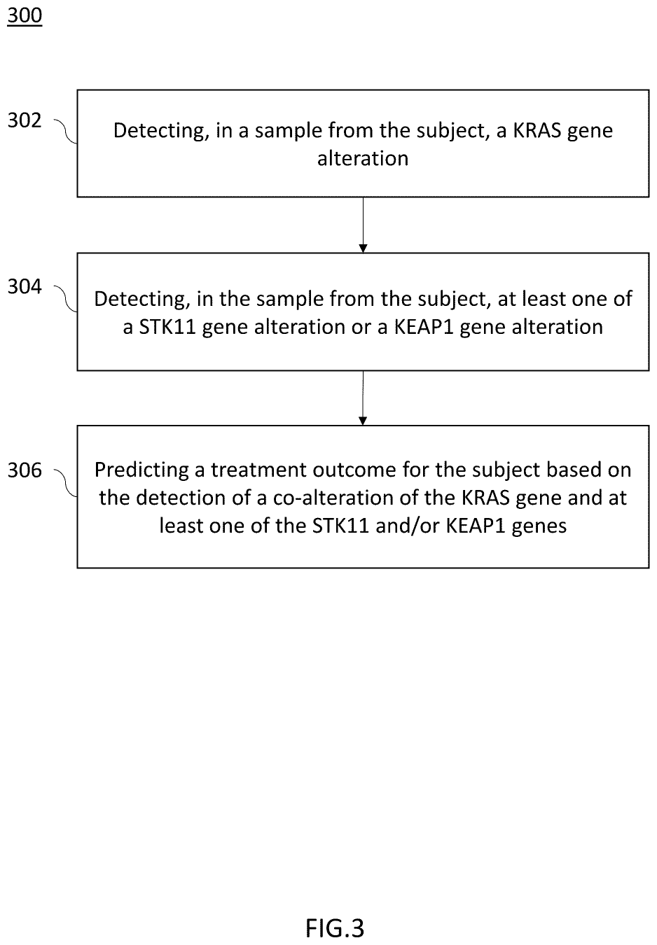

FIG. 3 provides a non-limiting example of a flowchart for a process 300 for predicting a treatment outcome for a subject. In some instances, the methods disclosed herein may be performed using sequencing-based and/or non-sequencing-based methods (e.g., microarray-based or PCR-based genotyping methods) for detection of KRAS, STK11, and KEAP1 gene alterations.

At step 302 in FIG. 3, a KRAS gene alteration-if present-is detected in the sample.

At step 304 in FIG. 3, at least one of a STK11 gene alteration or a KEAP1 gene alteration are detected-if present-in the sample.

In some instances, the detection of a KRAS gene alteration, a SYK11 gene alterations, and/or a KEAP1 gene alteration in the sample from the subject may comprise the use of a sequencing-based and/or non-sequencing-based method (e.g., a microarray-based or PCR-based genotyping method; see, e.g., Steuerwald et al. (2024), “Understanding the Biology and Testing Techniques for Pharmacogenomics in Oncology: A Practical Guide for the Clinician”, JCO Oncol. Pract. 20:1441-1461).

As noted above with respect to FIG. 1, in some instances, a gene alteration (e.g., a KRAS gene alteration, a STK11 gene alteration, or a KEAP1 gene alteration) may comprise a single nucleotide variant (SNV) (e.g., a single nucleotide substitution, a single nucleotide insertion, or a single nucleotide deletion), a short variant, an insertion or deletion (indel), a copy number variant (CNV), a rearrangement, a fusion, or a combination thereof.

In some instances, the KRAS gene alteration may comprise a KRAS short variant, a KRAS gene amplification, or a combination thereof. In some instances, the KRAS gene alteration may comprise a KRAS G12C alteration. In some instances, the STK11 and/or KEAP1 gene alterations may comprise a loss-of-function alteration.

At step 306 in FIG. 3, a treatment outcome is predicted for the subject based on the detection of a co-alteration of the KRAS gene and at least one of the STK11 and/or KEAP1 genes.

In some instances, the predicted treatment outcome for the subject is poor compared to that for a sample in which only a KRAS gene alteration is detected.

In some instances, the process may further comprise detecting at least one biomarker of genetic ancestry in the sample from the subject, and adjusting the predicted treatment outcome based on the detection of the at least one biomarker of genetic ancestry. In some instances, the at least one biomarker of genetic ancestry is indicative of European, African, East Asian, South Asian, and admixed American ancestry. In some instances, the at least one biomarker of genetic ancestry comprises a single nucleotide polymorphism (SNP)-based biomarker.

In some instances, the process may further comprise detecting a co-occurrence of a KRAS gene alteration and a GNAS gene alteration in a sample from a subject for which the at least one biomarker of genetic ancestry is indicative of admixed American ancestry, and adjusting the predicted treatment outcome based on the detection of the GNAS gene alteration.

In some instances, the process may further comprise detecting a co-occurrence of a KRAS gene alteration and an ARID1A gene alteration in a sample from a subject for which the at least one biomarker of genetic ancestry is indicative of South Asian ancestry, and adjusting the predicted treatment outcome based on the detection of the ARID1A gene alteration.

In some instances, the process may further comprise a tumor mutational burden (TMB) in a sample from a subject for which the at least one biomarker of genetic ancestry is indicative of African, and adjusting the predicted treatment outcome based on the determined TMB.

In some instances, the disease may be cancer. In some instances, the cancer may be non-squamous non-small cell lung cancer (non-Sq NSCLC).

In some instances, treatment of the disease may comprise treatment with an immune checkpoint inhibitor (ICI). In some instances, treatment of the disease may comprise treatment with a KRAS G12C inhibitor.

In some instances, in addition to, or instead of, predicting a treatment outcome, the method may comprise: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.

In some instances, a gene panel (e.g., for a targeted sequencing method or a microarray-based genotyping method) may comprise at least 1, at least 2, at least 3, at least 4, at least 5, at least 6, at least 7, at least 8, at least 9, at least 10, at least 20, at least 30, at least 40, at least 50, at least 75, at least 100, at least 125, at least 150, at least 175, at least 200, at least 250, at least 300, at least 350, or more than 350 genes.

In some instances, the disclosed methods may comprise the identification of variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRF11, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may may comprise the identification of variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods comprising the detection of a co-alteration of the KRAS gene and at least one of the STK11 and/or KEAP1 genes may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods comprising the detection of a co-alteration of the KRAS gene and at least one of the STK11 and/or KEAP1 genes may be used to select a subject (e.g., a patient) for a clinical trial. In some instances, patient selection for clinical trials based on, e.g., identification of a co-alteration of the KRAS gene and at least one of the STK11 and/or KEAP1 genes, may accelerate the development of targeted therapies and improve the healthcare outcomes for treatment decisions.

In some instances, the disclosed methods comprising the detection of a co-alteration of the KRAS gene and at least one of the STK11 and/or KEAP1 genes may be used to select an appropriate therapy or treatment (e.g., an anti-cancer therapy or anti-cancer treatment) for a subject. In some instances, for example, the anti-cancer therapy or treatment may comprise use of a poly ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erlcada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimctinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), clotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligco), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods comprising the detection of a co-alteration of the KRAS gene and at least one of the STK11 and/or KEAP1 genes may be used in treating a disease (e.g., a cancer) in a subject. For example, in response to detecting a co-alteration of the KRAS gene and at least one of the STK11 and/or KEAP1 genes using any of the methods disclosed herein, an effective amount of an anti-cancer therapy or anti-cancer treatment may be administered to the subject.

In some instances, the disclosed methods comprising the detection of a co-alteration of the KRAS gene and at least one of the STK11 and/or KEAP1 genes may be used for monitoring disease progression or recurrence (e.g., cancer or tumor progression or recurrence) in a subject. For example, in some instances, the methods may be used to detect a co-alteration of the KRAS gene and at least one of the STK11 and/or KEAP1 genes in a first sample obtained from the subject at a first time point, and used to detect a co-alteration of the KRAS gene and at least one of the STK11 and/or KEAP1 genes in a second sample obtained from the subject at a second time point, where comparison of the first determination and the second determination allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the detection of a co-alteration of the KRAS gene and at least one of the STK11 and/or KEAP1 genes.

In some instances, the identification of a co-alteration of the KRAS gene and at least one of the STK11 and/or KEA1 genes may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods comprising the detection of a co-alteration of the KRAS gene and at least one of the STK11 and/or KEAP1 genes may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods comprising the detection of a co-alteration of the KRAS gene and at least one of the STK11 and/or KEAP1 genes as part of a genomic profiling process (or inclusion of the output from the disclosed methods as part of the genomic profile of the subject) can improve the validity of, e.g., disease detection calls and treatment decisions, made on the basis of the genomic profile by, for example, independently confirming the presence of a disease in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypercosinophilic syndrome, systemic mastocytosis, familiar hypercosinophilia, chronic cosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROSI gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoictic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutancous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAcasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde-or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22):4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.R FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426.

Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence clements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiScq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (c) aligning said sequence reads using an alignment method as described elsewhere hercin; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (sec, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (sec, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5):589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011;21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (sec, e.g., SNVMix-Bioinformatics. 2010 Mar. 15; 26(6):730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the disclosed methods comprising the detection of a co-alteration of the KRAS gene and at least one of the STK11 and/or KEAP1 genes in a sample from a subject. The systems may comprise, e.g., one or more processors, and a memory unit communicatively coupled to the one or more processors and configured to store instructions that, when executed by the one or more processors, cause the system to: receive sequence read data for a plurality of sequence reads derived from a sample from a subject; detect a KRAS gene alteration in the sample from the subject based on the sequence read data; detect at least one of a STK11 gene alteration or a KEAP1 gene alteration in the sample from the subject based on the sequence read data; and diagnose or confirm a diagnosis of disease in the subject based on the detection of a co-alteration of the KRAS gene and at least one of the STK11 and/or KEAP1 genes.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for processing sequencing-based or non-sequencing-based genotyping data derived from any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the detection of a co-alteration of the KRAS gene and at least one of the STK11 and/or KEAP1 genes may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

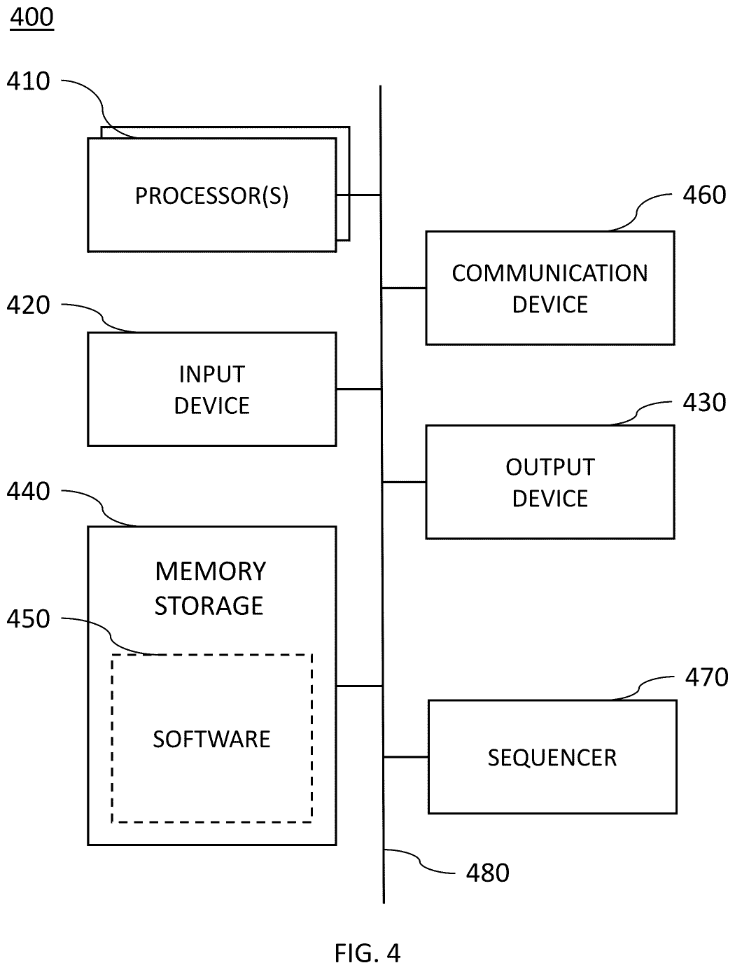

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

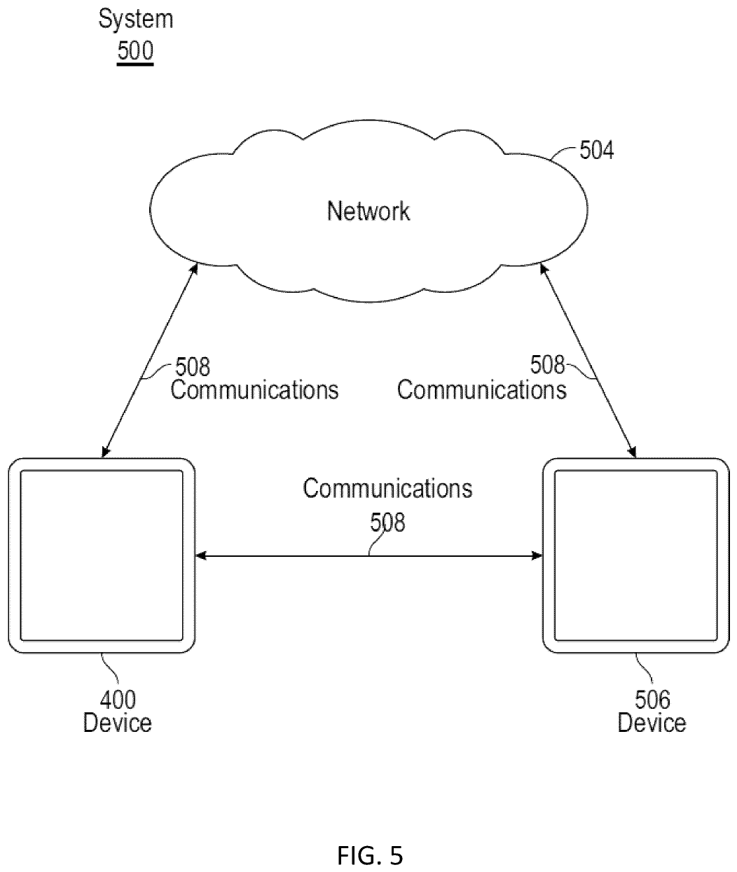

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG.4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, Pacific Biosciences' PacBio® RS system, Ultima Genomics UG 100™ platform, or the Illumina NovaSeq X series platform.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 1008, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXAMPLES

The following examples are included for illustrative purposes only and are not intended to limit the scope of the present disclosure.

### Example 1

### Ancestry-Associated Co-Alteration Landscape of KRAS and EGFR-Altered Non-Squamous NSCLC

### Abstract

Racial/ethnic disparities mar NSCLC care and treatment outcomes. While socioeconomic factors and access to healthcare are important drivers of NSCLC disparities, a deeper understanding of genetic ancestry-associated genomic landscapes can better inform the biology and the treatment actionability for these tumors. A comprehensive ancestry-based prevalence and co-alteration landscape of genomic alterations and immunotherapy-associated biomarkers in patients with KRAS and EGFR-altered non-squamous (non-Sq) NSCLC is presented. KRAS was the most frequently altered oncogene in European (EUR) and African (AFR), while EGFR alterations predominated in East Asian (EAS), South Asian (SAS), and Admixed American (AMR) groups, consistent with prior studies. As expected, STK11 and KEAP1 alterations co-occurred with KRAS alterations while showing mutual exclusivity with EGFR alterations. EAS and AMR KRAS-altered non-Sq NSCLC showed lower rates of co-occurring STK11 and KEAP1 alterations relative to other ancestry groups. Ancestry-specific co-alterations included co-occurrence of KRAS and GNAS alterations in AMR, KRAS and ARID1A alterations in SAS, and the mutual exclusivity of KRAS and NF1 alterations in the EUR and AFR ancestries. Contrastingly, EGFR-altered tumors exhibited a more conserved co-alteration landscape across ancestries. AFR exhibited the highest tumor mutational burden, with potential therapeutic implications for these tumors.

### Introduction

Lung cancer is the second most commonly diagnosed cancer globally and is the leading cause of cancer-related deaths. Non-Small Cell Lung Cancer (NSCLC) accounts for 80-85% of all lung cancers, with non-squamous (non-Sq) NSCLC, particularly lung adenocarcinoma, as the most common histologic subtype. Despite declines in NSCLC-related mortality in the past decade in the US, racial and ethnic disparities persist, including minority patient groups presenting with cancer at a younger age and more advanced stage compared to White individuals.

The advent of precision medicine and comprehensive genomic profiling using targeted sequencing has led to unique opportunities to identify the optimal treatment options for patients, requiring a growing emphasis on biomarker-driven clinical trials and a need to better define patient cohorts for these trials. As such, current drug development and patient enrollment in clinical trials are heavily influenced by our understanding of the prevalence of genomic alterations. However, due to the limited access to certain medical centers by minority groups and other historical biases, the databases utilized to inform the size of biomarker-selected patient populations are overwhelmingly composed of data from patients of Western European descent. Further, minority populations including Black, Asian, Hispanic/Latino, and Indigenous Peoples represent a small fraction of the patient population characterized in the widely utilized dataset, The Cancer Genome Atlas (TCGA) (12%, 3%, 3%, <0.5% respectively), and these populations continue to be underrepresented in clinical trials.

Genomic profiles, gene expression changes and prognostic significance of specific biomarkers have been shown to vary by race and ancestry across different tumor types, including NSCLC. However, interpretation of clinical trial outcomes and real-world implications, including identification of predictive and prognostic biomarkers, is heavily constrained in underrepresented populations. Due to a limited understanding of biomarker prevalence in patients from minority populations, forecasting clinical trial enrollment metrics to facilitate inclusive and equitable healthcare has also been hampered. Furthermore, because of their underrepresentation in clinical studies, fuller comprehension of the implications of the clinical data, including therapeutic indexes, pharmacokinetics and pharmacodynamics and drug safety and toxicity attributes of experimental treatments in the minority patient population groups has been challenging.

There are many contributing factors to the disparities observed in cancer outcomes and representation in clinical studies, including a general lack of trust in the medical establishment, limited awareness of cancer screening and clinical trial opportunities, long-standing effects of structural racism and environmental factors across different racial/ethnic groups. However, the magnitude of observed differences cannot solely be attributed to socioeconomic factors. Differences in the prevalence and the landscape of molecular alterations in different ancestry groups may also impact cancer outcomes and impact equitable representation in clinical trials. However, the continued lack of diversity in clinical studies has led to a rather poor understanding of the contribution of genomics to the disparities in the prevalence and outcomes of non-Sq NSCLC in different ancestry groups.

While self-reported race has been utilized to study the genetic features associated with cancer incidence and outcomes, this is often a challenge in clinical sequencing assays, where self-reported race and ethnicity information is not always available. However, using measures of genetic ancestry that can be derived from the data and provide accurate decipherment of ancestry for each patient, can play a critical role in understanding genetic basis of cancer disparities in different populations and provide insights on targetable therapies and precision medicine efforts in diverse populations. Here, the prevalence and co-alteration landscape of genomic alterations in non-Sq NSCLC was investigated using a diverse real-world cohort comprising patients of European (EUR), African (AFR), East Asian (EAS), South Asian (SAS) and Admixed American (AMR) ancestries, focusing the analyses on tumors with alterations in KRAS and EGFR, the two major oncogenic drivers of non-Sq NSCLC. Additionally, ancestry-associated patterns of programmed death-ligand 1 (PD-L1) expression and tumor mutational burden (TMB) are presented, known predictive biomarkers of response to immune checkpoint inhibitors, across the five ancestry groups to gain molecular insights into ancestry-based genomic landscapes as well as to better inform strategies for patient treatment and clinical trial enrollment.

### Methods

### Comprehensive Genomic Profiling Cohort of Non-Squamous NSCLC

The study cohort included 68,297 patients with non-squamous non-small cell lung cancer, who received comprehensive genomic profiling (CGP), as part of routine clinical care, through December 2022 from formalin-fixed, paraffin embedded (FFPE) tumor biopsies. Approval for this study, including a waiver of informed consent and a Health Insurance Portability and Accountability Act waiver of authorization, was obtained from the WCG Institutional Review Board. No clinical or treatment information was available for the samples in this cohort.

Briefly, DNA was extracted from FFPE sections and CGP was performed on hybridization-captured, adapter ligation-based libraries for exons of ˜324 (first CGP assay) and ˜315 (second CGP assay) cancer-related genes and select introns of genes frequently rearranged in cancer, as described previously. Data analysis was limited to 296 genes commonly targeted on both the assays. The reported genomic alterations included known or likely pathogenic short variants (base substitutions, small insertions/deletions), copy number alterations (gene amplifications of oncogenes, and homozygous deletions of tumor suppressors) as well as gene fusions and rearrangements predicted to activate oncogenes or inactivate tumor suppressors 63,64, and included a multi-step detection procedure described in detail in a prior study. Disease diagnosis included: lung adenocarcinoma (n=53,661), lung non-small cell lung carcinoma (nos) (n=12,533), lung large cell neuroendocrine carcinoma (n=1,246), lung sarcomatoid carcinoma (n=527), lung large cell carcinoma (n=276), lung carcinosarcoma (n=54).

### Prediction of Genetic Ancestry

The genetic ancestry for each patient was predicted using a single nucleotide polymorphism (SNP)-based approach, from the targeted next-generation sequencing assay (32396860, 37890492, 35176763). Briefly, >40,000 germline single nucleotide polymorphisms (SNPs) included in the targeted gene panel sequencing were overlapped with those captured in the phase 3 1000 Genomes Project and projected using principal component analysis to five principal components (PCs). These PCs were used to train a random forest classifier; the classifier performance was evaluated using ten-fold cross validation performed on the 1000 genomes project cohort, as described previously (37890492). Individuals were classified into the following ancestry groups based on the identified predominant ancestry: European, African, East Asian, South Asian, and admixed American. Comparison of genomic patterns in each ancestry group was performed using the European ancestry group as a reference.

### Detection of Immunotherapy-Related Biomarkers

Tumor mutational burden, calculated as the number of non-driver synonymous and non-synonymous mutations across a ˜0.8-1.2 megabase (Mb) region, using a prior validated approach 66 was also reported as part of the CGP profiling. In addition, data on PD-L1 expression was available for 29,945 cases. PD-L1 expression was determined by immunohistochemistry (IHC) performed on FFPE tissue sections using the Dako 22C3 PD-L1 antibody, according to the manufacturer's instructions (catalog number SK006). PD-L1 expression was binned into three categories based on the fraction of tumor cells staining with ≥1% intensity: negative (<1%), low positive (1-49%), or high positive (≥50%).

### Statistical Methods and Software

A comparison of the prevalence of gene alterations in the overall non-Sq NSCLC between the five ancestry groups was performed using a Chi-squared test. Similarly, to assess the difference in prevalence of each KRAS¬ and EGFR alteration identified between the five ancestry groups, a Chi-squared test was used. P-values were calculated for each comparison and adjusted for multiple hypothesis correction using the Benjamini-Hochberg false discovery rate (FDR) procedure. A Chi-squared test was also used to compare the breakdown of the three different alteration categories (short variant only, amplification only, and short variant+amplification) in KRAS- and EGFR¬-altered non-Sq NSCLCs within each ancestry group to their breakdown in the EUR ancestry group. Differences in prevalence of co-occurring gene alterations and immunotherapy-related biomarkers between ancestry groups in KRAS-and EGFR-altered NSCLCs were evaluated using a Fisher's exact test. This was also specifically applied for comparing the prevalence of concomitant KRAS, KEAP1, and STK11 alterations within each ancestry. Comparisons were made against the EUR group for each other ancestry subgroup. Two-sided P-values were calculated for each comparison and then adjusted for multiple hypothesis correction using the Benjamini-Hochberg FDR procedure. The distribution of TMB across the ancestry groups were compared using the Kruskal Wallis test, for the overall cohort as well as for KRAS- and EGFR¬-altered non-Sq NSCLCs. For each ancestry subgroup, patterns of co-occurrence and mutual exclusivity between KRAS and other targeted genes in the panel, as well as between EGFR and other targeted genes in the panel were also evaluated using a Fisher's exact test with FDR-based correction. Statistical significance was set at an FDR-corrected p≤0.05.

### Results

### KRAS and EGFR Alterations Show Varying Prevalence in Different Ancestry Groups

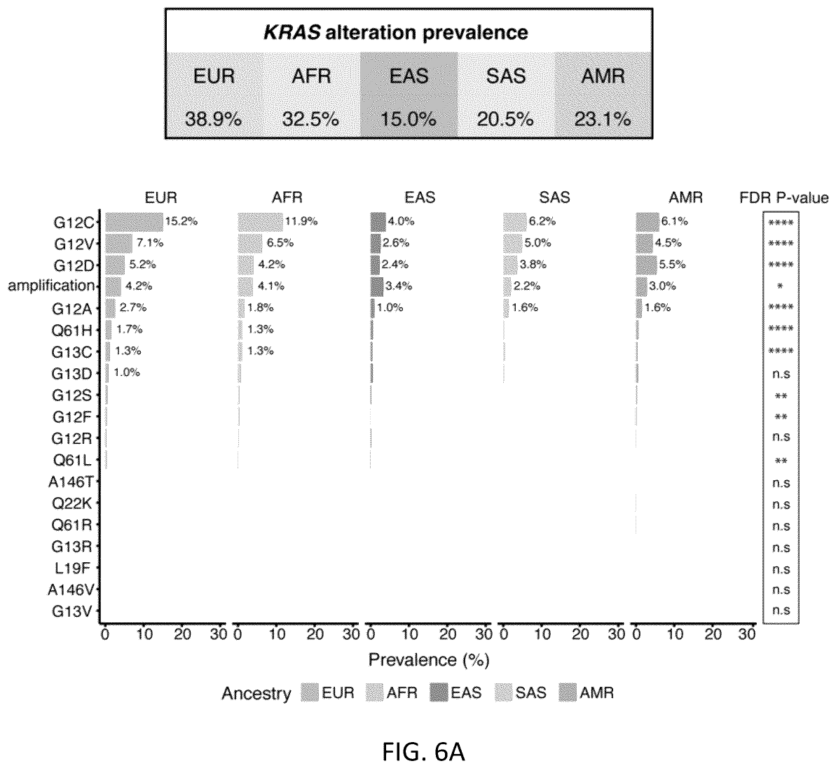

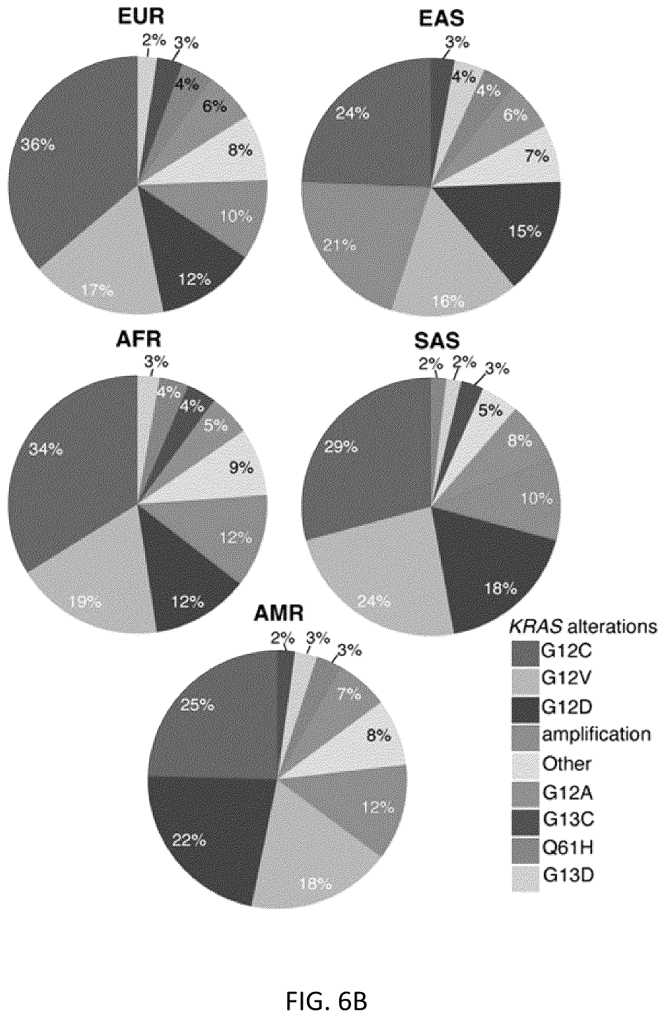

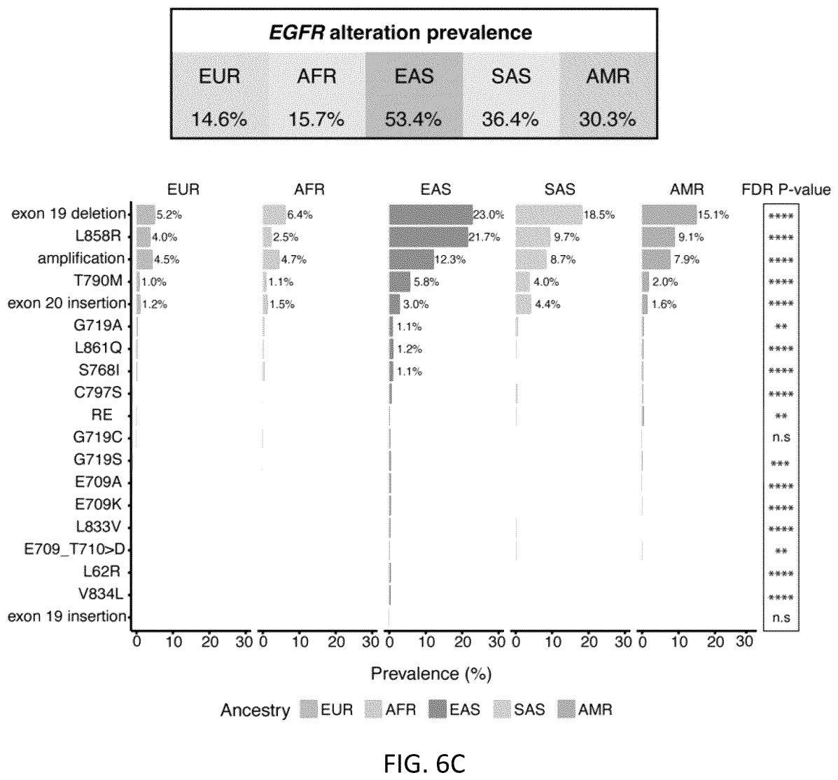

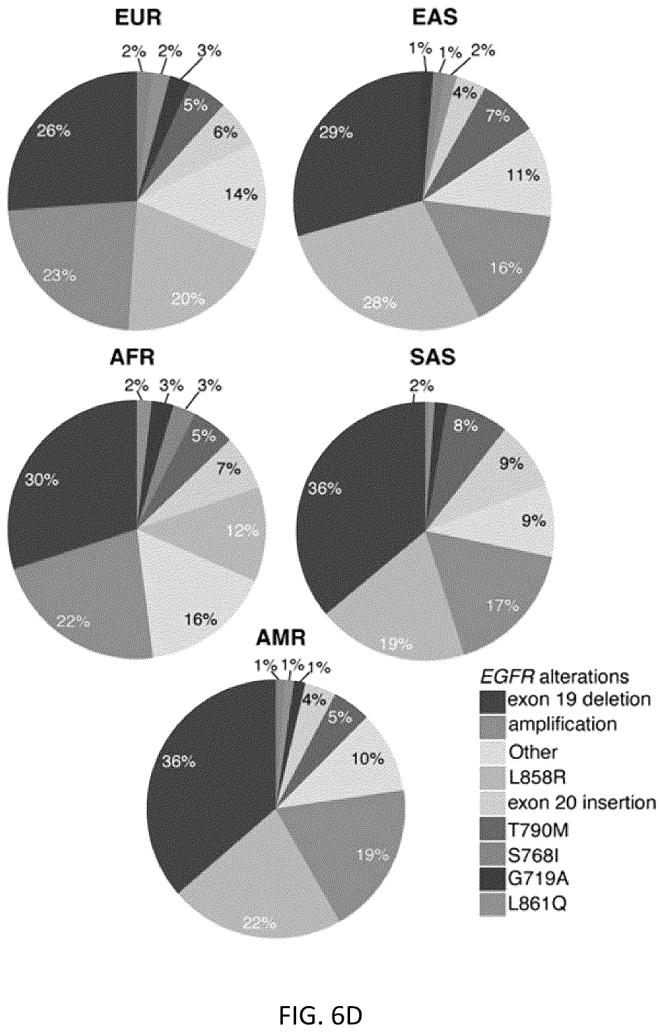

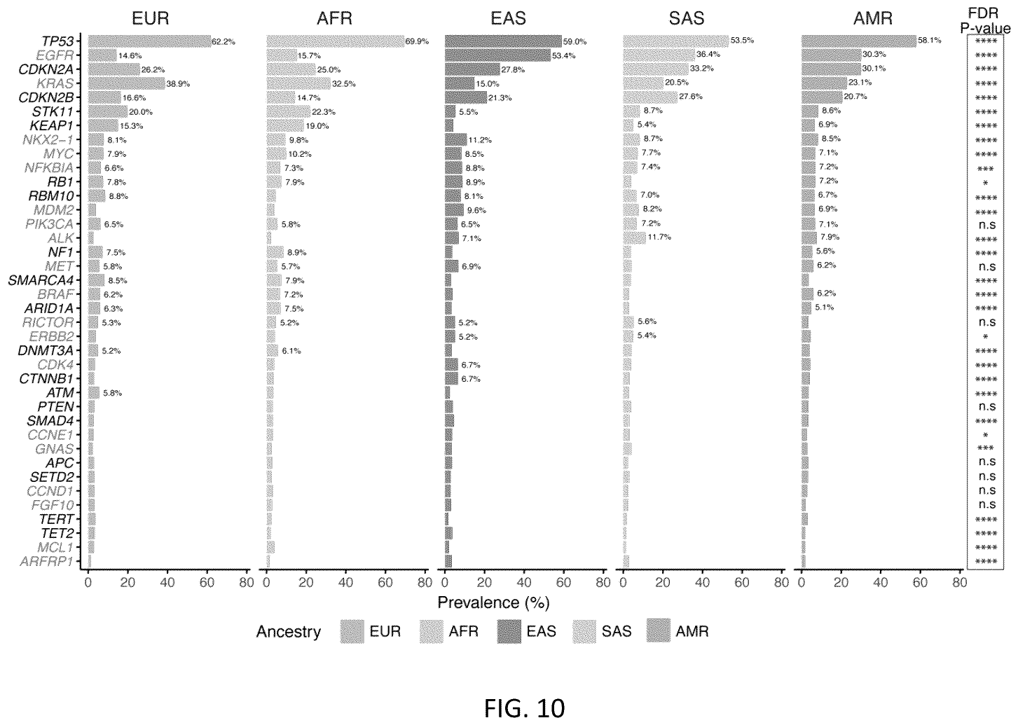

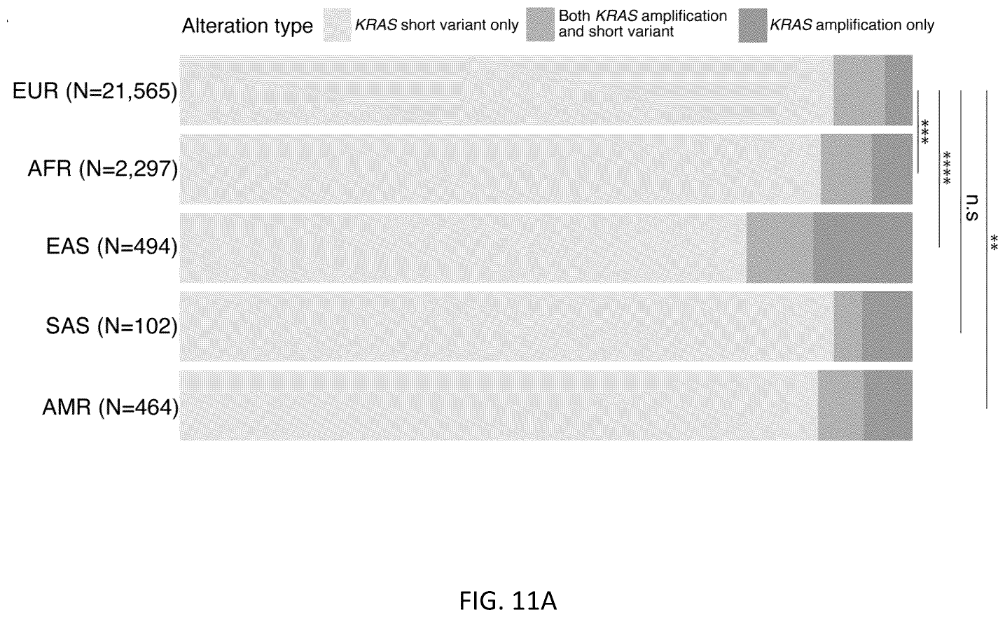

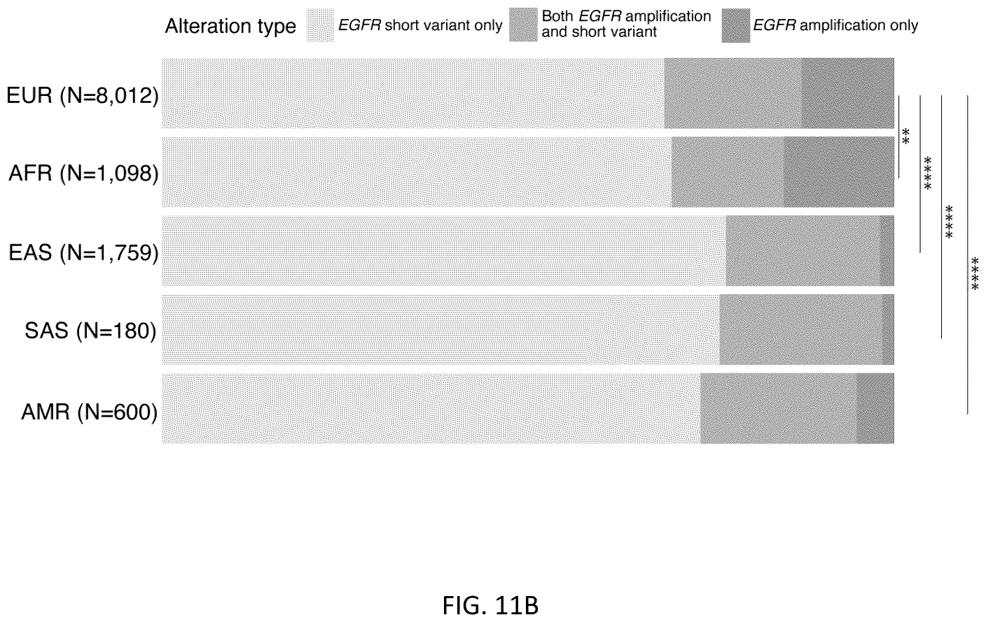

A total of 68,297 adult patients with non-Sq NSCLC who received tissue biopsy-based comprehensive genomic profiling (CGP) using two tissue-based assays (n =24,042 and n =44,255,respectively) during routine clinical care were included in this study. This US-based cohort included 55,430 patients of EUR ancestry (81%), 7062 patients of AFR ancestry (10%), 3297 patients of EAS ancestry (5%), 2011 patients of AMR ancestry (3%) and 497 patients of SAS ancestry (<1%). The overall genomic landscape of non-Sq NSCLC varied by ancestry (as depicted in FIG. 10). As expected, KRAS and EGFR were the most frequently altered oncogenes in the overall dataset 25, with additional ancestry-associated patterns. In EUR and AFR ancestry, KRAS was the most frequently altered oncogene (38.9% EUR, 32.5% AFR) followed by EGFR (14.6% EUR, 15.7% AFR) (FIG. 10). In contrast, for EAS, SAS as well as AMR subgroups, EGFR was the most frequently altered oncogene (53.4% EAS, 36.4% SAS, 30.3% AMR), with KRAS alterations being relatively less prevalent (15.0% EAS, 20.5% SAS, 23.1% AMR) (FIG. 10). KRAS G12C was the most prevalent KRAS alteration across all ancestry groups, with a higher prevalence in EUR (15.2%) and AFR (11.9%) groups compared to other ancestry groups (4-6%) (FIG. 6A). In comparison, KRAS G12D and G12V alterations, while rare in EAS (2.4%), were observed at similar rates (˜4-6%) across other ancestry groups (FIG. 6A). Notably, the significantly lower rate of KRAS G12C in EAS, SAS and AMR ancestry was the predominant contributor to the lower observed prevalence of overall KRAS alterations in these ancestry groups compared to EUR and AFR ancestry groups (FIG. 6B). In comparison to base substitutions, KRAS amplifications were less common (4.1% prevalence across ancestry groups). Interestingly KRAS amplifications represent 21% of all KRAS alterations in the EAS ancestry group, which is higher than the rates observed in the other ancestry groups (FIG. 6A, FIG. 6B). Of note, samples with KRAS amplifications frequently harbored concurrent KRAS short variants across all ancestrics; 65.1% EUR, 55.6% AFR, 48.3% AMR, 40.2% EAS and 36.4% SAS KRAS-amplified cases also had a KRAS short variant (see FIG. 11A below).

In contrast to KRAS, EGFR alterations were most commonly seen in patients of EAS ancestry (53.4%), with exon 19 deletions and L858R being the most prevalent EGFR alterations across all ancestry groups (FIG. 6C). While these two alteration groups occurred at similar rates in EAS and EUR populations (˜23-29% of all EGFR alterations), L858R alterations were less common than exon 19 deletions in SAS, AMR and AFR populations (˜19-22% vs. 30-38% of all EGFR alterations respectively; FIG. 6D). EGFR amplifications were also commonly observed across ancestry groups: 12.3% EAS, 8.7% SAS, 7.9% AMR, 4.7% AFR and 4.5% EUR (FIG. 6C). While EAS, SAS and AMR populations displayed a high prevalence of EGFR amplifications, >90% of EAS and SAS cases and >80% of AMR cases with EGFR amplifications had concomitant EGFR short variants (sec FIG. 11B below). In comparison, only 50-60% of EUR and AFR non-Sq NSCLC with EGFR amplification showed concomitant EGFR short variants (FIG. 11B). In addition to these alterations, exon 20 insertions, albeit overall rare, were more common in SAS population (4.4% of samples, ˜9% of all EGFR alterations) compared to other ancestry groups (FIG. 6C, FIG. 6D).

### Co-Alteration Landscape of KRAS and EGFR-Altered Non-Sq NSCLC Reveals Unique Ancestry-Specific Patterns

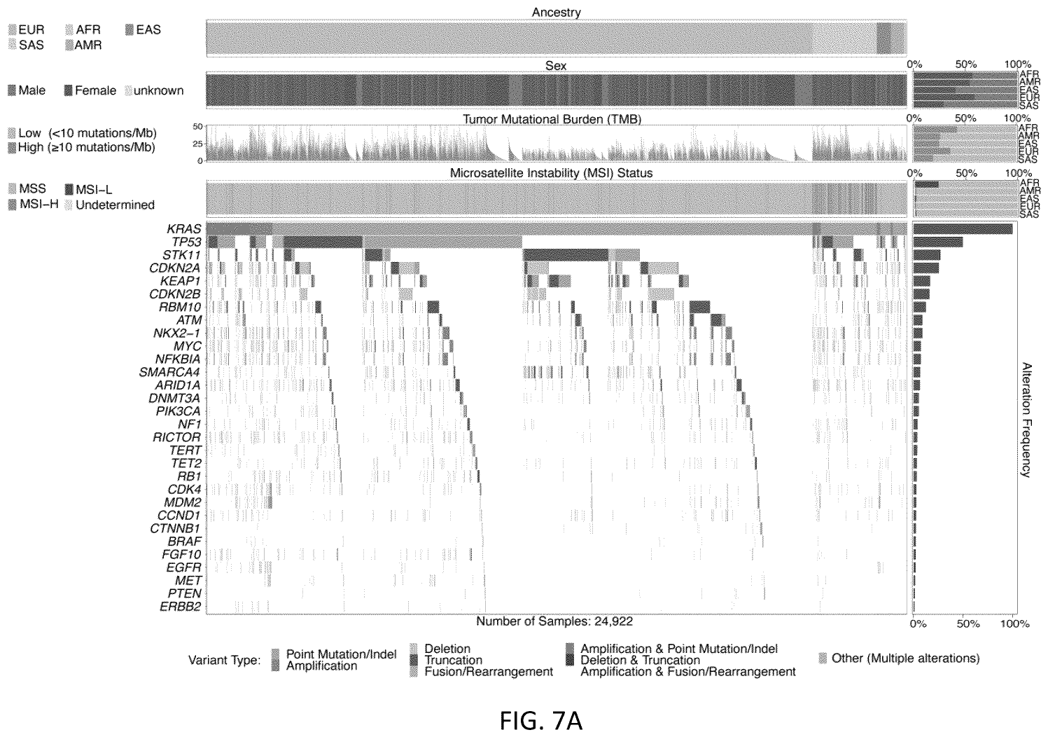

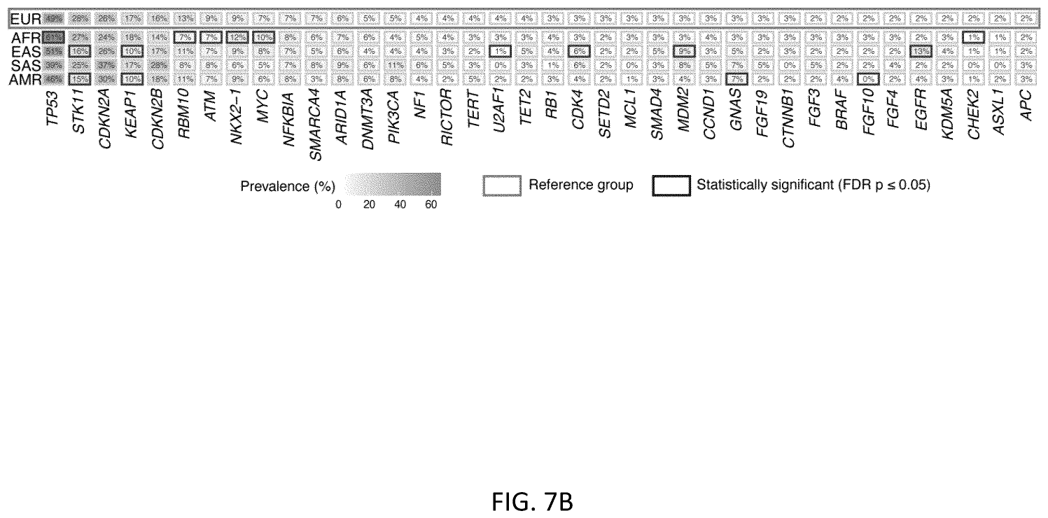

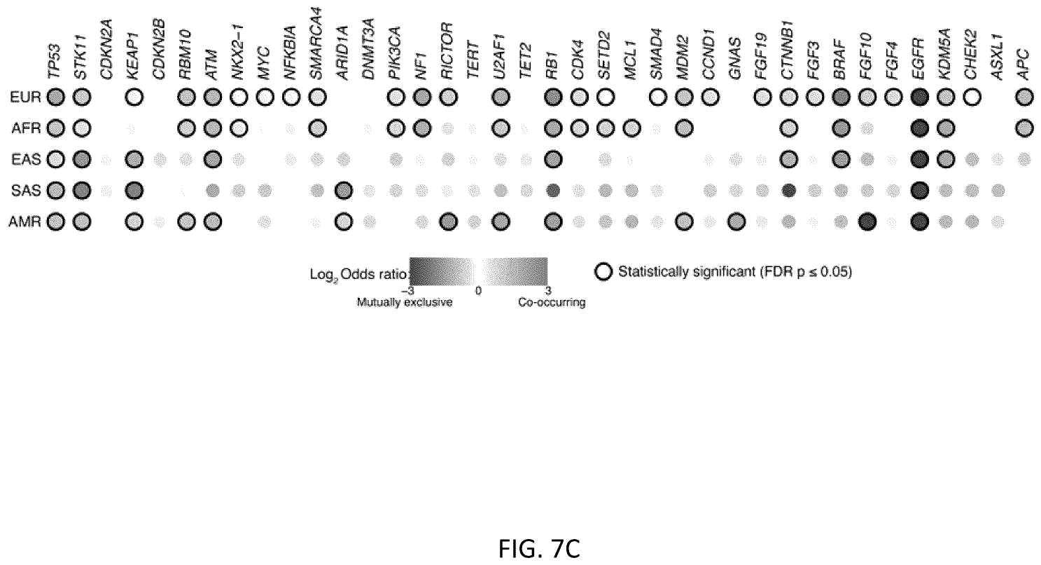

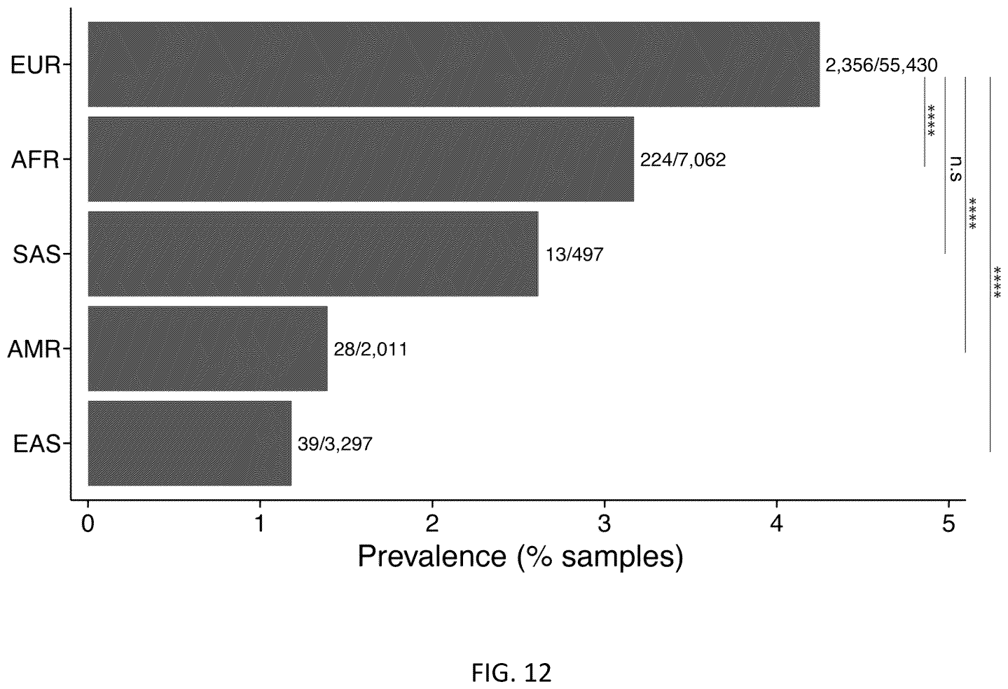

Among the 24,922 non-Sq NSCLC harboring KRAS alterations, the most common co-alterations included loss of function alterations in TP53, STK11, KEAP1 and CDKN2A/B (FIG. 7A). Interestingly, AFR showed a higher rate of TP53 co-alteration compared to EUR (60.7% vs. 48.8%, p<10-5; FIG. 7B). Despite TP53 being the most common alteration in KRAS-altered tumors, the prevalence was lower than in KRAS wildtype cases (48.8% vs. 70.7% EUR, 60.7% vs. 74.3% AFR, 51.4% vs. 60.4% EAS, 39.2% vs. 57.2% SAS, 45.9% vs. 61.8% AMR, all p<0.05) (FIG. 7C). Alterations in STK11 and KEAP1 showed strong co-occurrence with KRAS alterations across all ancestry groups (FIG. 7C). Interestingly, both STK11 and KEAP1 co-alterations were less common in EAS and AMR subgroups compared to EUR, AFR and SAS (FIG. 7B). Consistently, EAS and AMR also showed a lower prevalence of concomitant KRAS, STK11 and KEAP1 alterations (˜1% compared to 4.2% in EUR, see FIG. 12). As expected, KRAS alterations were mutually exclusive with alterations in other clinically actionable driver genes such as EGFR, ERBB2, RET, ALK, MET, ROSI and BRAF across all ancestries 26 (FIG. 7C).

Of note, despite the well-known mutual exclusivity of KRAS and EGFR alterations (FIG. 7C), 12.8% of the KRAS-altered tumors in the EAS ancestry showed co-occurring EGFR alterations as compared to 2-4% in the other ancestry groups (FIG. 7B). Upon closer examination, a vast majority of the 63 EAS cases with co-occurring EGFR and KRAS alterations had an amplification of at least one of these genes. Most commonly, 39 cases harboring EGFR L858R, or exon 19 deletions exhibited KRAS amplifications; 15 cases harbored amplifications in both EGFR and KRAS apart from short variants in one or more of these genes. The presence of amplifications in at least one of these genes may be explained by the synthetic lethality of concomitant KRAS and EGFR activating mutations. Additionally, the presence of concomitant EGFR and KRAS alterations may represent treatment resistance mechanisms within these tumors.

Co-occurring alterations in DNA damage checkpoint kinase (ATM), lysine-specific demethylase (KDM5A) and cyclin D2 (CCND2) were also noted in KRAS-altered tumors across most ancestries (FIG. 7B, FIG. 7C; not evaluable in SAS due to limited number of cases). Apart from alterations that were consistently co-occurring or mutually exclusive with KRAS alterations across ancestry groups, several genomic alterations were also found to be uniquely enriched or depleted in specific ancestries (FIG. 7C). For example, strong co-occurrence with KRAS alterations was observed for GNAS alterations in AMR (6.9% in KRAS(+) vs 2.1% in KRAS(−) cases; OR=3.5, p<10-5), ARID1A in SAS (8.9% in KRAS(+) vs 2.0% in KRAS(−) cases; OR=4.7, p=0.02), while mutual exclusivity of NF1 alterations was predominant in EUR (4.0% in KRAS(+) vs 9.7% in KRAS(−) cases; OR=0.4, p<10-5) and AFR (4.8% in KRAS(+) vs 10.8% in KRAS(−) cases; OR=0.4, p<10-5) ancestries (FIG. 7C). Of note, MDM2 alterations showed mutual exclusivity with KRAS alterations in EUR, AFR and AMR groups; interestingly, MDM2 alterations occurred in 8% of KRAS-altered SAS cases (p=0.63) and 9% EAS (p<10-5) compared to only 3% of KRAS-altered cases in EUR (FIG. 7B).

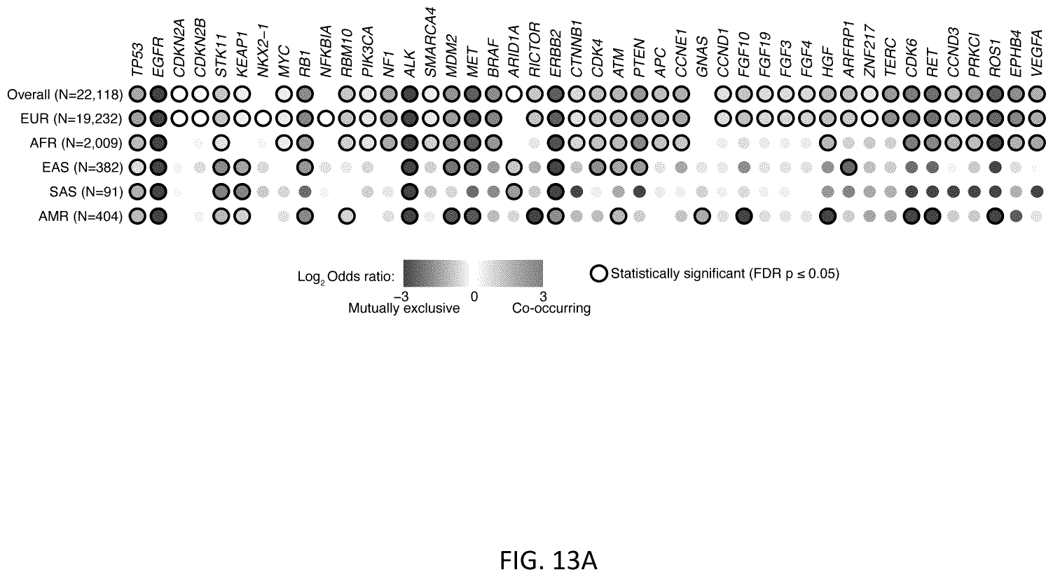

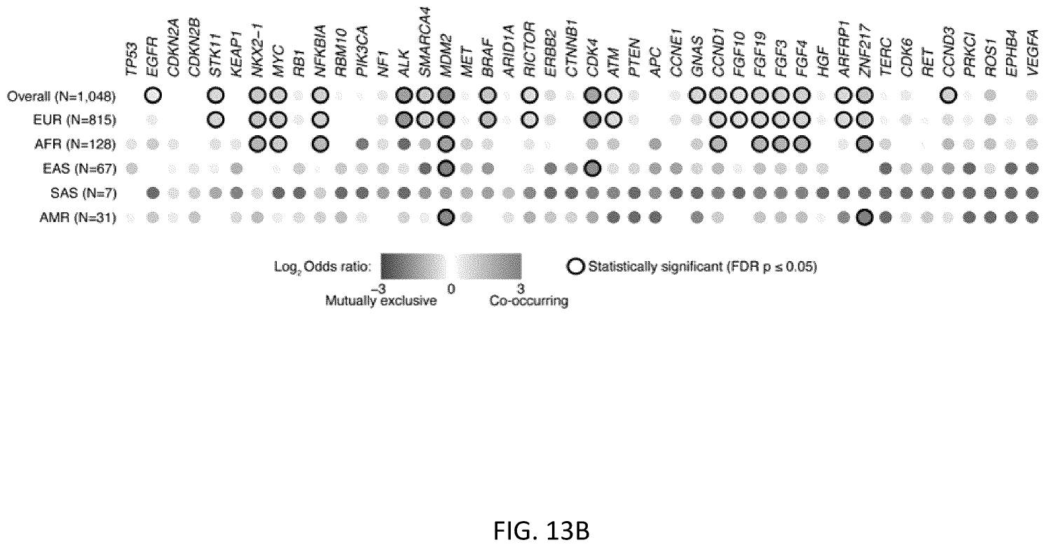

Given a prior observation of frequently co-occurring KRAS amplifications and short variants, the co-occurrence and mutual exclusivity profile of the samples exclusively harboring either KRAS short variants or KRAS amplifications was studied. Samples with KRAS short variants showed a higher degree of mutually exclusive alterations compared to KRAS amplified samples (sec FIG. 13A, FIG. 13B). For example, MDM2 alterations (amplifications) were mutually exclusive with KRAS short variants, while co-occurring with KRAS amplifications. A similar trend was observed for other alterations, including RICTOR, CDK4 and CCND1. Due to the limited sample size of KRAS amplified EAS, SAS, and AMR cases, the study was generally underpowered for statistical evaluation of similar comparisons within these ancestry groups.

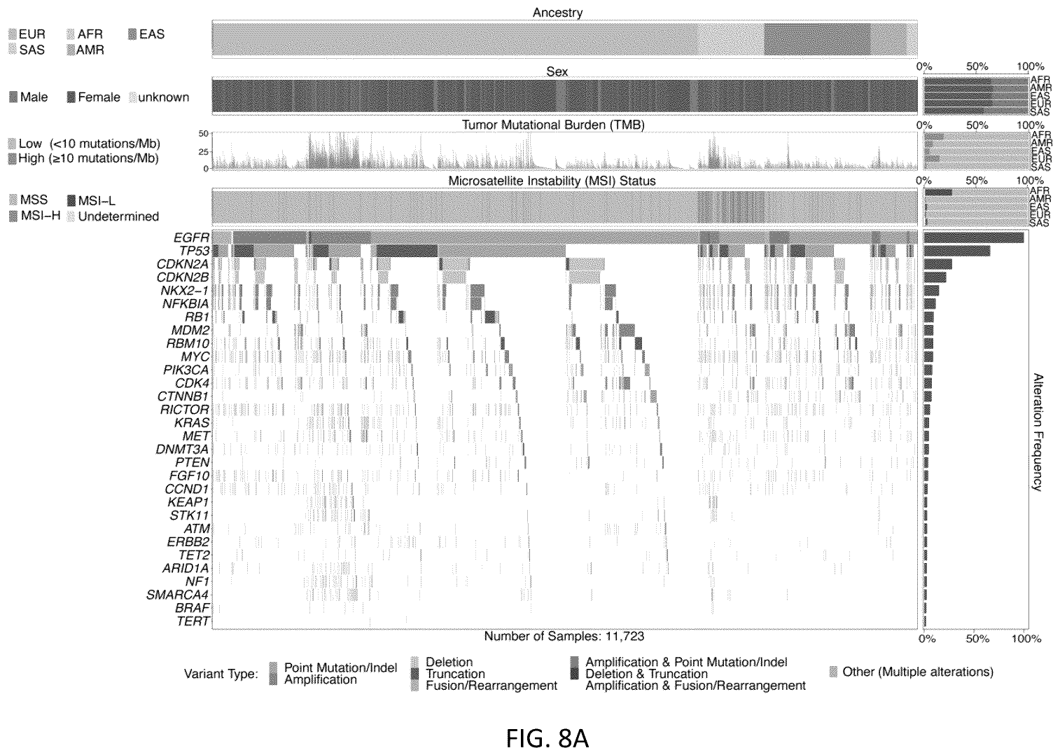

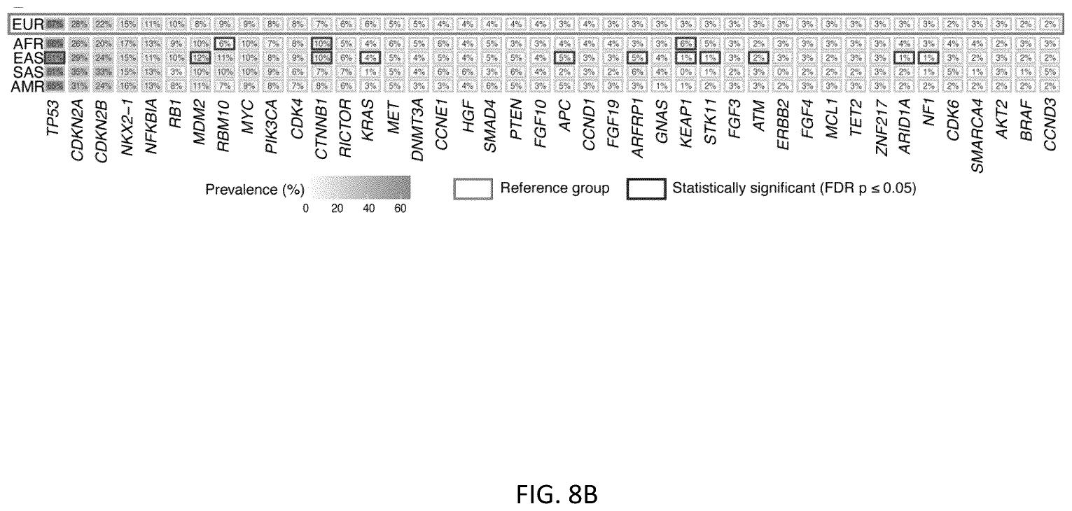

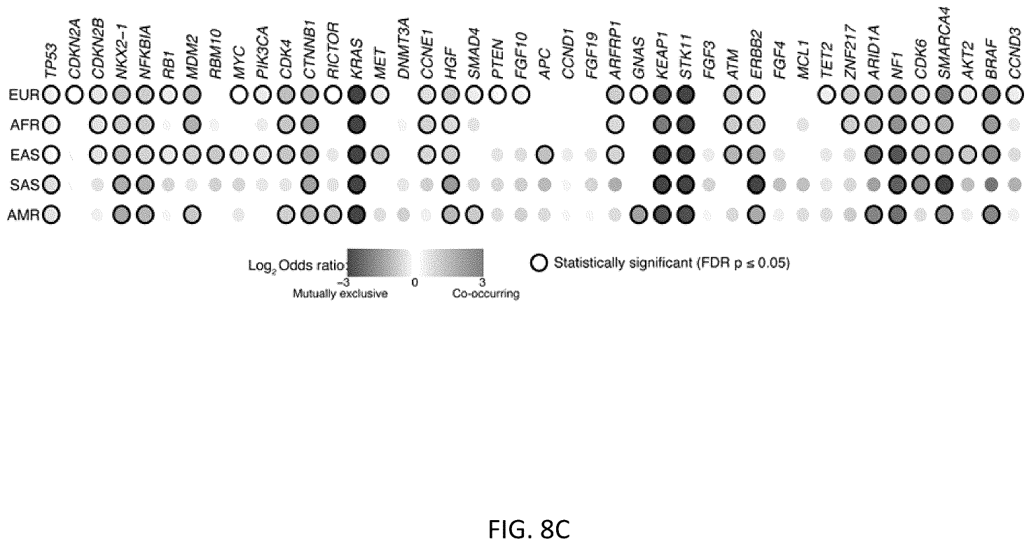

The co-alteration landscape of EGFR-altered tumors (FIG. 8A) was also interrogated. As with KRAS-altered tumors and owing to its high overall prevalence in non-Sq NSCLC, TP53 alterations were the most frequent in EGFR-mutant tumors across all ancestries (61-67%), followed by CDKN2A (26-31%) and CDKN2B alterations (24-33%) (FIG. 8A, FIG. 8B). STK11 and KEAP1 alterations that frequently co-occurred with KRAS mutations, showed strong mutual exclusivity with EGFR mutations across all ancestry groups (p<10-5) (FIG. 8C). Despite this general mutual exclusivity, EGFR-altered AFR cases showed a higher rate of KEAP1 co-alterations than EUR (6.1% vs. 3.3%, p=0.003), while EGFR-altered EAS cases showed a much lower rate of KEAP1 and STK11 alterations than EUR (˜1% vs ˜3%, p<10-5 for each) (FIG. 8B).

MDM2 alterations frequently co-occurred with EGFR alterations across ancestries (OR>2, p<10-5 for all groups except SAS; FIG. 8C); notably, MDM2 alterations were more frequent in EGFR-altered EAS as compared to the EGFR-altered EUR subgroup (12.3% vs 8.3%, p<10-5) (FIG. 8B), perhaps in part due to the higher overall prevalence of MDM2 alterations in the EAS population (see FIG. 10). Furthermore, in contrast to KRAS-altered tumors, ATM alterations showed mutual exclusivity with EGFR alterations in EUR, AFR and EAS ancestries (FIG. 8C). Consistent with prior studies portraying the importance of B-catenin signaling in EGFR-altered NSCLC 30, alterations in CTNNB1, the gene coding B-Catenin, which is the downstream effector of the Wnt signaling pathway, co-occurred with EGFR alterations across all ancestry groups, while being mutually exclusive with KRAS alterations (FIG. 7C, FIG. 8C).

Alterations in other genes such as NFKBIA, CDK6, CDK4 and CCNEI generally co-occurred with EGFR alterations across ancestry groups (FIG. 8C). Additionally, we also noted ancestry-specific patterns within EGFR-altered cases. For example, the EAS ancestry group showed high co-occurrence between EGFR and the Wnt pathway activating APC loss-of-function alterations (OR=2.5, p=2×10−5) (FIG. 8C). Amplifications of hepatocyte growth factor (HGF) gene, which encodes a known ligand of the RTK receptor MET, were seen in 4-6% of EGFR-altered cases and were found to co-occur with EGFR alterations across all ancestry groups (FIG. 8B, FIG. 8C). In general, co-occurring alterations in cell cycle regulatory genes, CDK4, CDK6, CCNEI and CCND3 were common in EGFR-altered tumors across different ancestry groups (FIG. 8B, FIG. 8C). In contrast and as noted above, most cell cycle gene alterations were mutually exclusive with KRAS alterations across ancestrics (FIG. 7C). NKX2-1, also known as thyroid transcription factor 1 (TTF-1), is a putative diagnostic marker in non-Sq NSCLC 31. Interestingly, amplifications in NKX2-1 showed significantly higher co-occurrence with EGFR as compared to KRAS alterations across all ancestries (FIG. 7C, FIG. 8C). As expected, amplifications of NFKBIA, present on the same amplicon as NKX2-1, also exhibited similar co-alteration and co-occurrence patterns (FIG. 7C, FIG. 8C). The observed NKX2-1 co-alteration pattern is supportive of the proposed differential functional significance of NKX2-1 in EGFR and KRAS mutated non-Sq NSCLC 32.

In contrast to KRAS-altered non-Sq NSCLC, EGFR-altered tumors appeared to have a more conserved co-occurrence and mutual exclusivity alteration landscape across ancestry groups (FIG. 7C, FIG. 8C). Expectedly, known oncogenic activating alterations in KRAS, BRAF, ALK and ROS1 were mutually exclusive with EGFR alterations, similar to the observation in KRAS-altered non-Sq NSCLC (FIG. 8C).

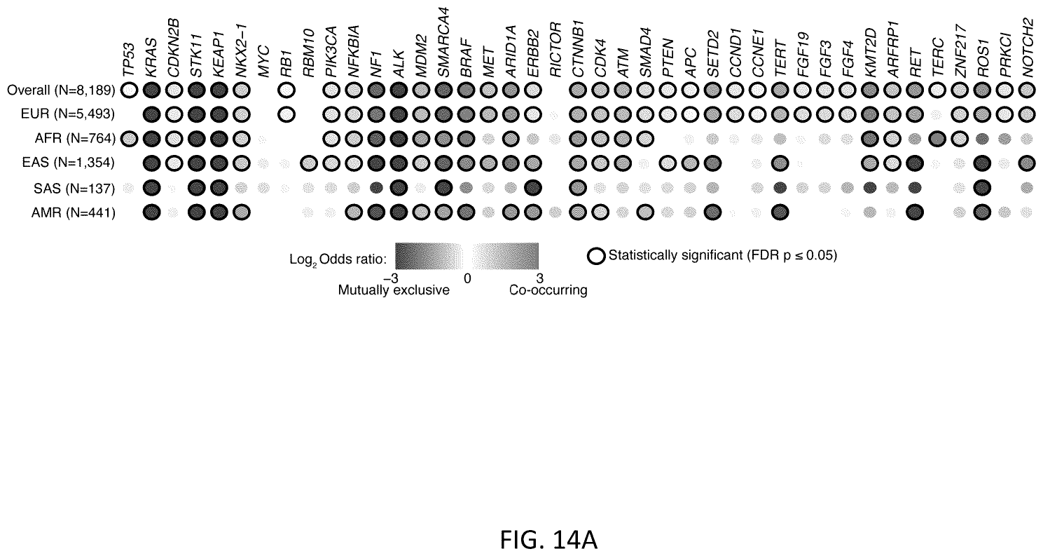

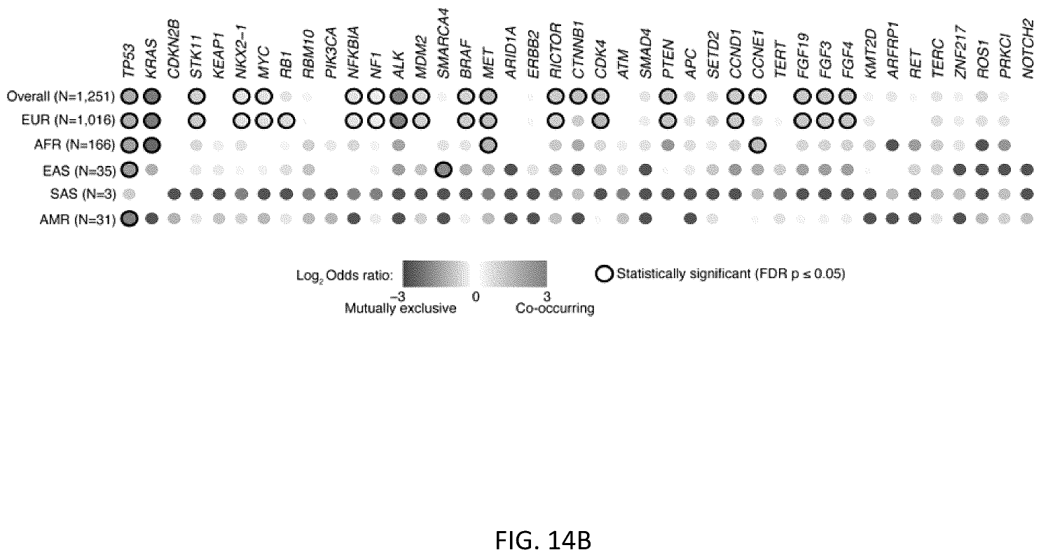

Based on the differential overlap of EGFR short variants and amplifications across ancestry groups noted previously (see FIG. 11B), the differences in the co-occurrence and mutual exclusivity profile of the samples exclusively harboring EGFR short variants or EGFR amplifications were also studied. EGFR amplified samples showed strong co-occurrence of TP53 alterations across all ancestry groups, while being mutually exclusive in cases with EGFR short variants, particularly in the AFR group (see FIG. 14A, FIG. 14B). Similarly, SMARCA4 alterations showed strong mutual exclusivity with EAS samples exclusively harboring EGFR short variants, while showing strong co-occurrence with EAS samples exclusively harboring EGFR amplifications (FIG. 14A, FIG. 14B). Additionally, alterations in NF1, MET, SMAD4, PTEN, amplifications of 11q13 comprising CCND1, FGF19, FGF3, and FGF4, also showed differing co-occurrence and mutually exclusive patterns in samples with EGFR short variants and EGFR amplification in specific ancestry groups (FIG. 14A, FIG. 14B).

Taken together, these data reveal a largely distinct co-alteration landscape between KRAS and EGFR-altered non-Sq NSCLC across ancestry groups and multiple overlapping as well as ancestry-specific co-occurring and mutually exclusive alterations within KRAS- and EGFR-altered non-Sq NSCLC.

### Immunotherapy-Associated Biomarkers Differ Based on Ancestry in KRAS and EGFR Altered Non-Sq NSCLC

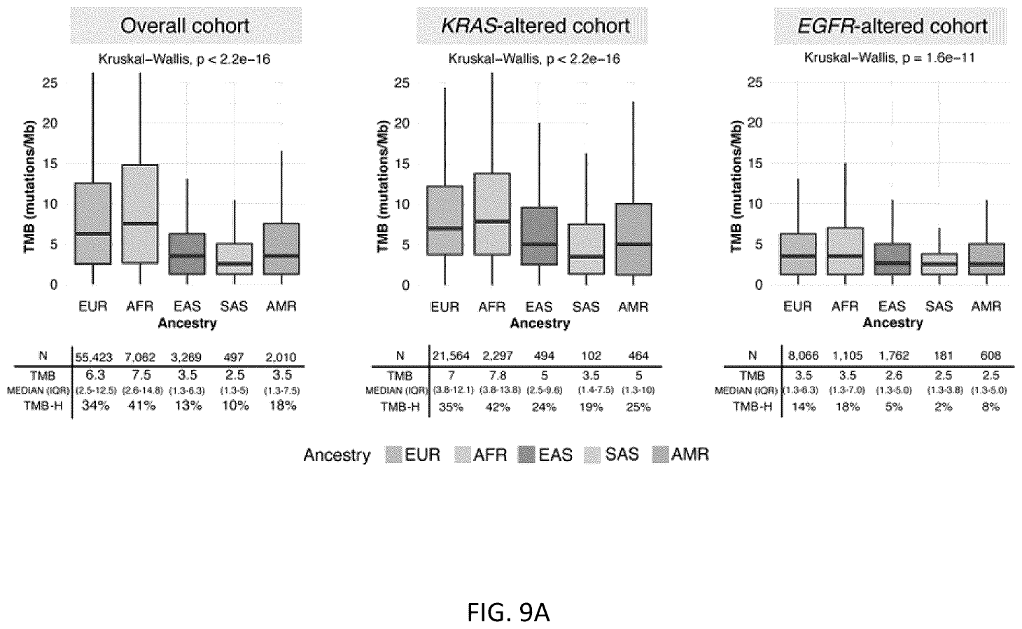

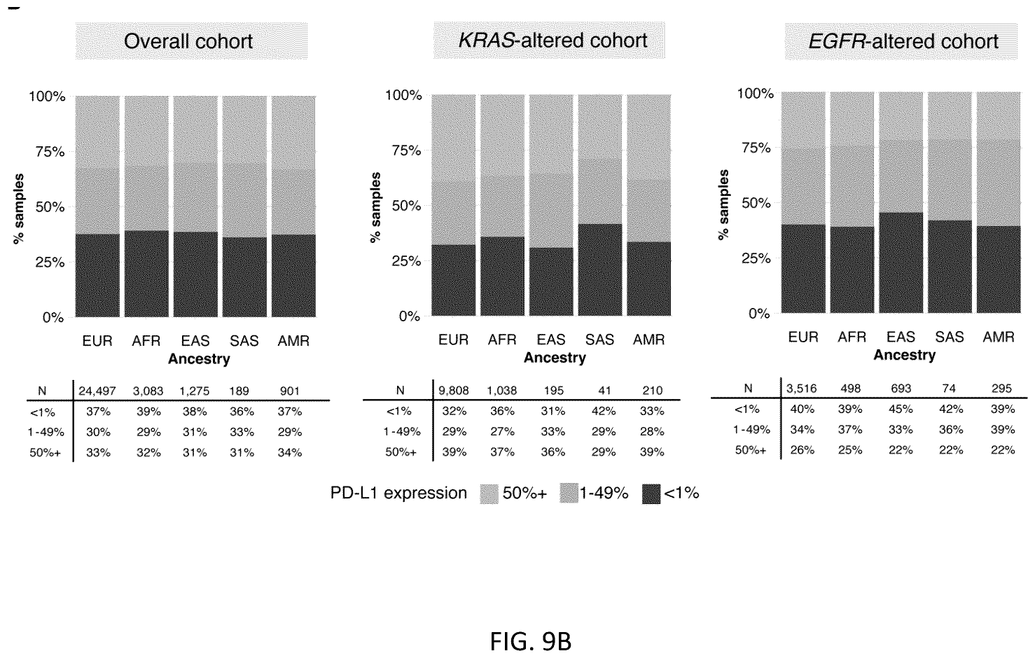

PD-L1/PDI signaling suppresses T cell activation and PD-L1 expression has emerged as a predictive biomarker for immune checkpoint inhibitors (ICI). High tumor mutational burden (TMB), associated with elevated neoantigen load, has also been proposed as a biomarker for potential benefit from ICI, with the FDA approval of pembrolizumab in unresectable or metastatic solid tumors with a TMB≥10 mutations/megabase (mut/Mb). Therefore, the relationship of these immunotherapy-associated biomarkers among KRAS- and EGFR-altered cases based on ancestry was explored. Across all non-Sq NSCLC, EUR and AFR showed a significantly higher TMB and a higher proportion of TMB-high cases (>10 mut/Mb) as compared to the EAS, SAS and AMR ancestries (34-41% vs. 10-18%; FIG. 9A, left). Comparing KRAS- and EGFR-altered cohorts, we observed a lower TMB in EGFR-altered cases, consistent with EGFR being more common among non-smokers (FIG. 9A, middle and right). Interestingly, even within KRAS- and EGFR-altered cohorts, we continued to observe a higher TMB in EUR and AFR compared to the other ancestry groups (FIG. 9A, middle and right). The AFR ancestry showed the highest proportion of TMB-high cases in the overall cohort (41%) as well as in KRAS-(42%) and EGFR-altered (18%) non-Sq NSCLC. In contrast, SAS exhibited the lowest proportion of TMB-high cases in the overall cohort (10%) and in KRAS-(19%) and EGFR-altered (2%) non-Sq NSCLC (FIG. 9A).

Despite the difference in TMB, the levels of PD-L1 were largely similar across ancestry groups, where approximately 30% of the overall non-Sq NSCLC cohort had PD-L1 expression of ≥50%, with an additional 30% showing expression levels 1-49% (FIG. 9B). Compared to KRAS-altered tumors, EGFR-altered tumors showed a lower percentage of PD-L1≥50% but a higher percentage of PD-L1 expression in 1-49% range and in the PD-L1 negative group (<1%), with largely similar overall patterns across ancestry groups (FIG. 9B, middle and right).

Overall, the higher proportion of tumors with TMB-high status and PD-L1≥50% in KRAS-altered cases compared to EGFR-altered cases may impact the potential therapeutic strategies within these genetic subgroups.

### Figure Descriptions

FIGS. 6A-C. Spectrum of KRAS and EGFR alterations in non-Sq NSCLC based on ancestry (EUR: European, AFR: African, EAS: East Asian, SAS: South Asian, AMR: Admixed American).

FIG. 6A provides a non-limiting example of data for the overall KRAS alteration prevalence (top) and the prevalence of individual KRAS alterations (bottom) in each ancestry group. Alterations observed in at least 50 cases in the overall non-Sq NSCLC cohort are shown. Statistically significant patterns, determined by a Chi-squared test followed by FDR correction are shown with the following P-value thresholds: * 0.05, ** 0.01, *** 0.001, **** 0.0001, n.s, not significant.

FIG. 6B provides a non-limiting example of pie charts displaying the breakdown of the different KRAS alterations observed in each ancestry group. The color and percentages denote the fraction of specific KRAS alterations relative to all the KRAS alterations detected.

FIG. 6C provides a non-limiting example of the overall EGFR alteration prevalence (top) and the prevalence of individual EGFR alterations (bottom) in each ancestry group. Alterations observed in at least 50 cases across all ancestry groups are shown. Statistically significant patterns, determined by a Chi-squared test followed by FDR correction, are shown with the following P-value thresholds: * 0.05, ** 0.01, *** 0.001, **** 0.0001, n.s, not significant.

FIG. 6D provides a non-limiting example of pie charts displaying the breakdown of the different EGFR alterations observed in each ancestry group. The color and percentages denote the fraction of specific EGFR alterations relative to all the KRAS alterations detected.

FIGS. 7A-C. Co-alteration landscape of KRAS-altered non-Sq NSCLC based on ancestry (EUR: European, AFR: African, EAS: East Asian, SAS: South Asian, AMR: Admixed American).

FIG. 7A provides a non-limiting example of a tileplot showing the overall mutational spectrum of KRAS-altered non-Sq NSCLC with alterations in the top 30 most frequently occurring genes displayed. Patterns of tumor mutational burden (TMB), microsatellite instability (MSI) and sample-level annotations of ancestry and sex are annotated. The gene alterations within a sample are colored based on the type of alteration.

FIG. 7B provides a non-limiting example of data illustrating the comparative prevalence of co-occurring gene alterations in KRAS-altered non-Sq NSCLC based on ancestry. Genes with a prevalence of at least 2% across all KRAS-altered cases are shown. The prevalence of a gene alteration in each ancestry was compared against European ancestry. Statistically significant patterns, determined by a Fisher's exact test with FDR correction (FDR p≤0.05), are highlighted with a black box.

FIG. 7C provides a non-limiting example of data illustrating patterns of co-occurrence and mutual exclusivity between KRAS and other gene alterations in each ancestry. Genes with a prevalence of at least 2% across all KRAS-altered cases are shown. Statistically significant patterns, determined by a Fisher's exact test with FDR correction (FDR p<0.05), are highlighted with a black circle. Patterns of co-occurrence are shown in shades of red (light gray) while patterns of mutual exclusivity are shown in shades of blue (dark gray).

FIGS. 8A-C. Co-alteration landscape of EGFR-altered non-Sq NSCLC based on ancestry (EUR: European, AFR: African, EAS: East Asian, SAS: South Asian, AMR: Admixed American).

FIG. 8A provides a non-limiting example of a tileplot showing the overall mutational spectrum of EGFR-altered non-Sq NSCLC with alterations in the top 30 most frequently occurring genes displayed. Patterns of tumor mutational burden (TMB), microsatellite instability (MSI) and sample-level annotations of ancestry and sex are annotated. The gene alterations within a sample are colored based on the type of alteration.

FIG. 8B provides a non-limiting example of data illustrating the comparative prevalence of co-occurring gene alterations in EGFR-altered non-Sq NSCLC based on ancestry. Genes with a prevalence of at least 2% across all EGFR-altered cases are shown. The prevalence of a gene alteration in each ancestry was compared against European ancestry. Statistically significant patterns, determined by a Fisher's exact test with FDR correction (FDR p≤0.05), are highlighted with a black box.

FIG. 8C provides a non-limiting example of data illustrating patterns of co-occurrence and mutual exclusivity between EGFR and other gene alterations in each ancestry. Genes with a prevalence of at least 2% across all EGFR-altered cases are shown. Statistically significant patterns, determined by a Fisher's exact test with FDR correction (FDR p≤0.05), are highlighted with a black circle. Patterns of co-occurrence are shown in shades of red (light gray) while patterns of mutual exclusivity are shown in shades of blue (dark gray).

FIGS. 9A-B. Patterns of immunotherapy associated biomarkers, overall, and in KRAS and EGFR-altered non-Sq NSCLC based on ancestry (EUR: European, AFR: African, EAS: East Asian, SAS: South Asian, AMR: Admixed American).

FIG. 9A provides a non-limiting example of data illustrating patterns of tumor mutational burden in the overall cohort, KRAS-altered and EGFR-altered non-Sq NSCLC based on ancestry. The color denotes the ancestry group. The total number of samples, the median TMB with the interquartile range (IQR) and the percentage of TMB-High (≥10 mutations/Mb) cases within each ancestry are also provided. Each box plot displays the interquartile range (IQR), with the lower and upper boundaries representing the 25th and 75th percentile; the line within the box represents the median and the whiskers extend to ±1.5×IQR.

FIG. 9B provides a non-limiting example of data illustrating patterns of PD-L1 positivity from DAKO 22C3 in the overall cohort, KRAS-altered and EGFR-altered non-Sq NSCLC based on ancestry. The color denotes the level of PD-L1 positivity: 50%+, 1-49% and 0%. The total number of samples with available PD-L1 information and the percentage of PD-L1 positive cases within each ancestry are also provided.

FIG. 10. Prevalence of gene alterations in the overall non-Sq NSCLC cohort based on ancestry. FIG. 10 provides a non-limiting example of a barplot showing the prevalence of the top gene alterations across different ancestry subgroups in the overall non-Sq NSCLC cohort based on ancestry. A union of the 30 most common gene alterations in each ancestry group, accounting for a total of 38 genes is plotted. The color of each bar denotes the ancestry group. Genes predicted to be oncogenes are shown in pink (light gray). (EUR: European, AFR: African, EAS: East Asian, SAS: South Asian, AMR: Admixed American). Statistically significant patterns, determined by a Chi-squared test followed by FDR correction, are shown with the following P-value thresholds: * 0.05, ** 0.01, *** 0.001, **** 0.0001, n.s, not significant.

FIGS. 11A-B. Overlap of short variants and amplifications within KRAS and EGFR in non-Sq NSCLC.

FIG. 11A provides a non-limiting example of data illustrating the breakdown of cases with KRAS short variants only, amplifications only, and cases with both classes of alterations within each ancestry group. The total number of KRAS-altered cases within each ancestry group is provided. Statistically significant patterns, determined by a Chi-squared test, are shown with the following P-value thresholds: * 0.05, ** 0.01, *** 0.001, **** 0.0001, n.s, not significant.

FIG. 11B provides a non-limiting example of data illustrating the breakdown of cases with EGFR short variants only, amplifications only, and cases with both classes of alterations within each ancestry group. The total number of EGFR-altered cases within each ancestry group is provided. Statistically significant patterns, determined by a Chi-squared test, are shown with the following P-value thresholds: * 0.05, ** 0.01, *** 0.001, **** 0.0001, n.s, not significant.

FIG. 12. Prevalence of concomitant KRAS, KEAP1 and STK11 alterations across ancestry groups. FIG. 12 provides a non-limiting example of a barplot displaying the prevalence of cases, as a percentage of total cases (N), with co-occurring alterations in KRAS, KEAP1, and STK11 in cach ancestry group. (EUR: European, AFR: African, EAS: East Asian, SAS: South Asian, AMR: Admixed American). Statistically significant patterns, determined by a Fisher's exact test, are shown with the following P-value thresholds: * 0.05, ** 0.01, *** 0.001, **** 0.0001, n.s, not significant.

FIGS. 13A-B. Co-occurrence and mutual exclusivity of gene alterations in cases with KRAS short variants and KRAS amplifications exclusively, across ancestry groups.

FIG. 13A provides a non-limiting example of data illustrating patterns of co-occurrence and mutual exclusivity between KRAS short variants and other gene alterations in each ancestry. Statistically significant patterns, determined by a Fisher's exact test with FDR correction (FDR p≤0.05), are highlighted with a black circle. Patterns of co-occurrence are shown in shades of red (light gray) while patterns of mutual exclusivity are shown in shades of blue (dark gray).

FIG. 13B provides a non-limiting example of data illustrating patterns of co-occurrence and mutual exclusivity between KRAS amplifications and other gene alterations in each ancestry. The same set of genes shown in panel A are shown here. Statistically significant patterns, determined by a Fisher's exact test with FDR correction (FDR p<0.05), are highlighted with a black circle. Patterns of co-occurrence are shown in shades of red (light gray) while patterns of mutual exclusivity are shown in shades of blue (dark gray).

Cases with co-occurring KRAS amplifications and short variants were excluded from these analyses. (EUR: European, AFR: African, EAS: East Asian, SAS: South Asian, AMR: Admixed American). Gene alterations showing statistically significant co-occurrence/mutual exclusivity with either KRAS short variants only or KRAS amplifications only, in at least one ancestry and with evaluable data across all ancestry groups are plotted.

FIG. 14A provides a non-limiting example of data illustrating patterns of co-occurrence and mutual exclusivity between EGFR short variants and other gene alterations in each of five different ancestry groups. Statistically significant patterns, determined by a Fisher's exact test with FDR correction (FDR p≤0.05), are highlighted with a black circle. Patterns of co-occurrence are shown in shades of red (light gray) while patterns of mutual exclusivity are shown in shades of blue (dark gray).

FIG. 14B provides a non-limiting example of data illustrating patterns of co-occurrence and mutual exclusivity between EGFR amplifications and other gene alterations in each of five different ancestry groups. Statistically significant patterns, determined by a Fisher's exact test with FDR correction (FDR p≤0.05), are highlighted with a black circle. Patterns of co-occurrence are shown in shades of red (light gray) while patterns of mutual exclusivity are shown in shades of blue (dark gray).

Cases with co-occurring EGFR amplifications and short variants were excluded from these analyses. (EUR: European, AFR: African, EAS: East Asian, SAS: South Asian, AMR: Admixed American). Gene alterations showing statistically significant co-occurrence/mutual exclusivity with either KRAS short variants only or KRAS amplifications only, in at least one ancestry and with evaluable data across all ancestry groups are plotted.

### Discussion

Previous studies by our group and others have presented the landscape and the therapeutic relevance of allele-specific KRAS and EGFR alterations in non-sq NSCLC; yet, these studies have lacked or had limited information on ancestry-specific patterns. Moreover, previous estimates of mutational prevalence, especially in minority patient populations, have been constrained by limited cohort size. Through the examination of a large and diverse cohort, we provide unequivocal confirmation for previously published prevalence metrics, including the higher prevalence of KRAS alterations in EUR and AFR ancestries compared to EAS, SAS and AMR ancestry groups and a higher prevalence of EGFR alterations in EAS, SAS and AMR compared to EUR and AFR ancestries, while offering new insights on ancestry-specific patterns of gene alterations and immunotherapy-associated biomarkers.

Although smoking is generally associated with KRAS-altered but not EGFR-altered non-Sq NSCLC, other factors, beyond environmental exposures, may also contribute to the lower prevalence of KRAS alterations in EAS, SAS and AMR ancestries. Supporting this, EGFR mutations are shown to be consistently more prevalent than KRAS mutations in the EAS ancestry regardless of smoking status. Ancestral differences in the prevalence of EGFR and KRAS alterations are also driven by underlying ancestry-specific germline differences. This is supported by higher EGFR and lower KRAS mutation prevalence in Native American (NAT) and Hispanic/Latino populations, together comprising the AMR ancestry group, which includes components of the EAS ancestry, likely derived through waves of Asian-Pacific migration. Moreover, independent of smoking status, NAT ancestry was shown to be associated with EGFR mutations. The observed lower prevalence of STK11 and KEAP1 alterations in the KRAS-altered EAS and AMR as compared to the other ancestry groups may also stem from ancestry-specific germline variations, as both NAT and Hispanic/Latino NSCLC populations have been shown to exhibit a relatively lower prevalence of STK11 and KEAP1 alterations compared to EUR and Non-Hispanic White patients. Interestingly, the SAS ancestry group appeared closer to EAS and AMR in the prevalence of EGFR and KRAS alterations, but showed STK11 and KEAP1 co-alterations in KRAS-altered non-Sq NSCLC at a rate similar to those scen in EUR and AFR.

STK11 and KEAP1 alterations individually or together are poor prognostic factors in NSCLC and an enrichment of STK11 and KEAP1 alterations in KRAS-mutant non-Sq NSCLC has been previously reported. Yet, the prevalence of these alterations in different ancestry groups had remained understudied. The significantly lower co-alteration rate of STK11 and KEAP1 in KRAS-altered EAS and AMR as compared to other ancestries raises questions regarding potential clinical implications, including treatment outcomes with ICI and the recently emerging KRAS G12C inhibitors as discussed below. The differential co-mutation profile of KRAS G12C mutant NSCLC tumors should be an important consideration when comparing clinical outcomes of different KRAS G12C inhibitors conducted in different regions of the world. It is noteworthy that the Asian patient population appears to derive a greater benefit from ICI as compared to non-Asian patients. While different lifestyles, environmental exposures, and general physiological differences in different ancestry groups may contribute to variable treatment outcomes, a distinct co-alteration landscape in EAS compared to EUR and AFR ancestries may partly underlie a generally better prognosis in the former patient population. Moreover, although unlike PD-L1 levels, the predictive value of TMB for the use of ICI remains debated, high mutational burden has been shown as a poor prognostic factor, and EAS non-Sq NSCLC have been shown to harbor more stable genomes as compared to EUR non-Sq NSCLC. Together with a lower STK11 and KEAP1 co-alteration rate, higher genomic stability of the KRAS-mutant EAS ancestry group may explain better treatment outcomes compared to EUR and AFR ancestries. Conversely, no differential benefit with EGFR inhibitors has been observed in Asian patients with EGFR-mutant cancer compared to non-Asians 56, which is consistent with a more conserved co-alteration landscape in EGFR-mutant non-Sq NSCLC across ancestries.

Targeted inhibition of mutant EGFR utilizing highly efficacious third-generation EGFR inhibitors has transformed patient care in NSCLC. Moreover, a subset of KRAS-altered non-Sq NSCLC, representing the biggest alteration subgroup in White and Black patient populations, has long been un-druggable but is now starting to benefit from mutant-specific KRAS G12C inhibitors. However, unlike EGFR-mutant non-Sq NSCLC that derive impressive clinical benefit from EGFR inhibitors, thus far, KRAS G12C inhibitors have shown relatively less pronounced clinical activity in KRAS G12C-mutant non-Sq NSCLC. A higher overall mutation burden and less conserved co-alteration landscape may partly explain this disparity in clinical activity, and it remains to be seen how distinct co-alteration patterns in KRAS G12C mutant NSCLC across ancestries may influence the activity of KRAS G12C inhibitors. This underscores the importance of assessing the co-alteration landscape of these tumors and studying the potential impact of ancestry on clinical outcomes to KRAS G12C inhibitors.

Co-occurring KEAP1, CDKN2A and SMARCA4 loss-of-function alterations have recently emerged as poor prognostic biomarkers in KRAS G12C-mutant non-Sq NSCLC treated with the KRAS G12C inhibitors, sotorasib and adagrasib. While SMARCA4 alterations distributed rather evenly across the five ancestry groups studied here, a lower KEAP1 co-alteration rate in EAS and AMR may associate with better treatment outcomes in these patient populations. Although CDKN2A alterations generally appear to be a poor prognostic factor, associated with worse outcomes not only with KRAS G12C inhibitors but also ICI and EGFR inhibitors, the observed similar prevalence of CDKN2A co-alterations in both KRAS and EGFR-mutant non-Sq NSCLC across ancestry groups is unlikely to impact treatment outcomes across ancestry groups. These findings warrant further work to understand the impact of co-occurring alterations on the efficacy of different treatment modalities, especially in AFR, AMR and SAS ancestries, which remain vastly understudied.

With a higher prevalence of actionable RTK alterations, EAS, SAS, and AMR might harbor a larger actionable population over EUR and AFR, deriving clinical benefit from multiple approved and effective RTK inhibitors. However, with a bigger proportion of PD-L1≥50% tumors, the KRAS-mutant EUR and AFR non-Sq NSCLC may derive additional benefit from ICI. Despite a relatively higher proportion of PD-L1≥50% tumors, higher STK11 and KEAP1 co-alterations in KRAS-mutant EUR and AFR are expected to attenuate the activity of ICI and therapy in general. Together, these findings unveil a complex web of molecular alterations that may impact therapeutic susceptibility of KRAS and EGFR-mutant non-Sq NSCLC across different ancestry groups and consequently may contribute to molecular underpinnings of cancer disparities. A limitation of this study is the missing underlying clinical outcome data and treatment history, with the latter bearing possible implications for the observed genomic alteration landscape. Although the analysis included more than 300 cancer-relevant genes, additional parallels, and differences in the co-alteration landscape of KRAS and EGFR-mutant non-Sq NSCLC and across different ancestries are likely to continue to emerge along with future comprehensive sequencing efforts and evolving treatment landscape.

In summary, by informing the prevalence of KRAS and EGFR alterations, the associated genomic co-alterations, and patterns of immunotherapy-associated biomarkers among different ancestry groups, the findings offer the potential to help formulate new therapeutic hypotheses, propel evaluation of new therapeutic strategies, and influence public health policies that may aid alleviation of cancer disparities.

### EXEMPLARY IMPLEMENTATIONS

Exemplary implementations of the methods and systems described herein include:
1. A method for diagnosing or confirming a diagnosis of disease in a subject, the method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
receiving, using one or more processors, sequence read data for the plurality of sequence reads;
detecting, using the one or more processors, a KRAS gene alteration in the sample from the subject based on the sequence read data;
detecting, using the one or more processors, at least one of a STK11 gene alteration or a KEAP1 gene alteration in the sample from the subject based on the sequence read data; and diagnosing or confirming a diagnosis of disease in the subject based on the detection of a co-alteration of the KRAS gene and at least one of the STK11 and/or KEAP1 genes.
2. A method for predicting a treatment outcome for a subject diagnosed with or suspected of having a disease, the method comprising:
   detecting, in a sample from the subject, a KRAS gene alteration;
   detecting, in the sample from the subject, at least one of a STK11 gene alteration or a KEAP1 gene alteration; and
   predicting a treatment outcome for the subject based on the detection of a co-alteration of the KRAS gene and at least one of the STK11 and/or KEAP1 genes.
3. The method of clause 2, wherein the disease is cancer, and optionally, wherein the cancer is non-squamous non-small cell lung cancer (NSCLC).
4. The method of clause 2 or clause 3, wherein the predicted outcome for the subject is poor compared to that for a sample in which only a KRAS gene alteration is detected.
5. The method of any one of clauses 2 to 4, further comprising detecting at least one biomarker of genetic ancestry in the sample from the subject, and adjusting the predicted treatment outcome based on the detection of the at least one biomarker of genetic ancestry.
6. The method of clause 5, wherein the at least one biomarker of genetic ancestry is indicative of European, African, East Asian, South Asian, and admixed American ancestry.
7. The method of clause 5 or clause 6, wherein the at least one biomarker of genetic ancestry comprises a single nucleotide polymorphism (SNP)-based biomarker.
8. The method of any one of clauses 5 to 7, further comprising detecting a co-occurrence of a KRAS gene alteration and a GNAS gene alteration in a sample from a subject for which the at least one biomarker of genetic ancestry is indicative of admixed American ancestry, and adjusting the predicted treatment outcome based on the detection of the GNAS gene alteration.
9. The method of any one of clauses 5 to 8, further comprising detecting a co-occurrence of a KRAS gene alteration and an ARID1A gene alteration in a sample from a subject for which the at least one biomarker of genetic ancestry is indicative of South Asian ancestry, and adjusting the predicted treatment outcome based on the detection of the ARID1A gene alteration.
10. The method of any one of clauses 5 to 9, further comprising a tumor mutational burden (TMB) in a sample from a subject for which the at least one biomarker of genetic ancestry is indicative of African, and adjusting the predicted treatment outcome based on the determined TMB.
11. The method of any one of clauses 1 to 10, wherein the KRAS gene alteration comprises a KRAS short variant, a KRAS gene amplification, or any combination thereof.
12. The method of any one of clauses 1 to 11, wherein the KRAS gene alteration comprises a KRAS G12C alteration.
13. The method of any one of clauses 1 to 12, wherein the STK11 and/or KEAP1 gene alterations comprise loss-of-function alterations.
14. The method of any one of clauses 1 to 13, wherein the disease is cancer.
15. The method of clause 14, wherein the cancer is non-squamous non-small cell lung cancer (NSCLC).
16. The method of any one of clauses 1 to 15, wherein treatment of the disease comprises treatment with an immune checkpoint inhibitor (ICI).
17. The method of any one of clauses 1 to 16, wherein treatment of the disease comprises treatment with a KRAS G12C inhibitor.
18. The method of any one of clauses 1 to 17, further comprising: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.
19. A system comprising:
   one or more processors; and
   a memory communicatively coupled to the one or more processors and configured to store instructions that, when executed by the one or more processors, cause the system to: receive sequence read data for a plurality of sequence reads derived from a sample from a subject;
   detect a KRAS gene alteration in the sample from the subject based on the sequence read data;
   detect at least one of a STK11 gene alteration or a KEAP1 gene alteration in the sample from the subject based on the sequence read data; and diagnose or confirm a diagnosis of disease in the subject based on the detection of a co-alteration of the KRAS gene and at least one of the STK11 and/or KEAP1 genes.
20. A non-transitory computer-readable storage medium storing one or more programs, the one or more programs comprising instructions, which when executed by one or more processors of a system, cause the system to:
receive sequence read data for a plurality of sequence reads derived from a sample from a subject;
detect a KRAS gene alteration in the sample from the subject based on the sequence read data;
detect at least one of a STK11 gene alteration or a KEAP1 gene alteration in the sample from the subject based on the sequence read data; and
diagnose or confirm a diagnosis of disease in the subject based on the detection of a co-alteration of the KRAS gene and at least one of the STK11 and/or KEAP1 genes.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for diagnosing or confirming a diagnosis of disease in a subject, the method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, using one or more processors, sequence read data for the plurality of sequence reads;
detecting, using the one or more processors, a KRAS gene alteration in the sample from the subject based on the sequence read data;
detecting, using the one or more processors, at least one of a STK11 gene alteration or a KEAP1 gene alteration in the sample from the subject based on the sequence read data; and
diagnosing or confirming a diagnosis of disease in the subject based on the detection of a co-alteration of the KRAS gene and at least one of the STK11 and/or KEAP1 genes.

2. A method for predicting a treatment outcome for a subject diagnosed with or suspected of having a disease, the method comprising:
detecting, in a sample from the subject, a KRAS gene alteration;
detecting, in the sample from the subject, at least one of a STK11 gene alteration or a KEAP1 gene alteration; and
predicting a treatment outcome for the subject based on the detection of a co-alteration of the KRAS gene and at least one of the STK11 and/or KEAP1 genes.

3. The method of claim 2, wherein the disease is cancer, and optionally, wherein the cancer is non-squamous non-small cell lung cancer (NSCLC).

4. The method of claim 2, wherein the predicted outcome for the subject is poor compared to that for a sample in which only a KRAS gene alteration is detected.

5. The method of claim 2, further comprising detecting at least one biomarker of genetic ancestry in the sample from the subject, and adjusting the predicted treatment outcome based on the detection of the at least one biomarker of genetic ancestry.

6. The method of claim 5, wherein the at least one biomarker of genetic ancestry is indicative of European, African, East Asian, South Asian, and admixed American ancestry.

7. The method of claim 5, wherein the at least one biomarker of genetic ancestry comprises a single nucleotide polymorphism (SNP)-based biomarker.

8. The method of claim 5, further comprising detecting a co-occurrence of a KRAS gene alteration and a GNAS gene alteration in a sample from a subject for which the at least one biomarker of genetic ancestry is indicative of admixed American ancestry, and adjusting the predicted treatment outcome based on the detection of the GNAS gene alteration.

9. The method of claim 5, further comprising detecting a co-occurrence of a KRAS gene alteration and an ARID1A gene alteration in a sample from a subject for which the at least one biomarker of genetic ancestry is indicative of South Asian ancestry, and adjusting the predicted treatment outcome based on the detection of the ARID1A gene alteration.

10. The method of claim 5, further comprising a tumor mutational burden (TMB) in a sample from a subject for which the at least one biomarker of genetic ancestry is indicative of African, and adjusting the predicted treatment outcome based on the determined TMB.

11. The method of claim 1, wherein the KRAS gene alteration comprises a KRAS short variant, a KRAS gene amplification, or any combination thereof.

12. The method of claim 1, wherein the KRAS gene alteration comprises a KRAS G12C alteration.

13. The method of claim 1, wherein the STK11 and/or KEAP1 gene alterations comprise loss-of-function alterations.

14. The method of claim 1, wherein the disease is cancer.

15. The method of claim 14, wherein the cancer is non-squamous non-small cell lung cancer (NSCLC).

16. The method of claim 1, wherein treatment of the disease comprises treatment with an immune checkpoint inhibitor (ICI).

17. The method of claim 1, wherein treatment of the disease comprises treatment with a KRAS G12C inhibitor.

18. The method of claim 1, further comprising: (i) identifying the subject for treatment of a disease, (ii) predicting a prognosis for the subject, (iii) selecting a treatment for the subject, (iv) treating the subject, (v) adjusting a treatment dose for the subject, (vi) identifying the subject for inclusion in a clinical trial, or (vii) monitoring the disease progression or recurrence in the subject, based on the detection of a co-alteration of the KRAS gene and at least one of the STK11 and/or KEAP1 genes.

19. A system comprising:
one or more processors; and
a memory communicatively coupled to the one or more processors and configured to store instructions that, when executed by the one or more processors, cause the system to:
receive sequence read data for a plurality of sequence reads derived from a sample from a subject;
detect a KRAS gene alteration in the sample from the subject based on the sequence read data;
detect at least one of a STK11 gene alteration or a KEAP1 gene alteration in the sample from the subject based on the sequence read data; and
diagnose or confirm a diagnosis of disease in the subject based on the detection of a co-alteration of the KRAS gene and at least one of the STK11 and/or KEAP1 genes.
